SEQUENCE LISTING (5 sequences)

SEQ ID NO: 1 - protein, 138 aa, Pleurotus ostreatus
MAYAQWVIIIIHNVGSQDVKIKNLKASWGKLHADGDKDAEVSASNYEGKIVKPDEKLQINACGRSDAAEGTTGTFDLVDPADGDKQVRHFYWDCPWGSKTNTWTVSGSNTKWMIEYSGQNLDSGALGTITVDTLKKGN

SEQ ID NO: 2 - protein, 138 aa, Pleurotus eryngii
MAYAQWVIIIIHNVGSKDVKIVNLKPSWGKLHADGDKDTEVSASKYEGTVIKPDEKLQINACGRSDAAEGTTGTFDLVDPADGDKQVRHFYWDCPWGSKANTWTVSGSNTKWMIEYSGQNLDSGALGTITVDTLKKGN

SEQ ID NO: 3 - protein, 138 aa, Pleurotus eryngii
MAYAQWVIILIHNVGQQNVKIKNLNASWGKLYADGDKDTEVPASKYEGMVIAPDDQVQINACGREDAAEGTTGTFDLVDPNDSDKQVRHFAWDCPWGTKANSWVVGGSNSKWMIEYTGQNLDSGALGTITVNTLRIGN

SEQ ID NO: 4 - protein, 523 aa, Pleurotus ostreatus
MEAVLSRQAATAEAIGRFQDSSTSVGLVAGSPSTRIRRQADNVVLKSTSQAGDTLNDVIQDPTRRNKLINDNNLLKGIIMGRDGPVPSSRELIVRPDTLRAIINNRATIETTTMEAEFTETLMESNYNSASVKVSAPFITANSEYSESSSFKNTETEKSMYTSSRYLFPQGRIDFTTPDSGFDDVIKLSPQFTSGVQAALAKATGTEKREALQNLFQEYGHVFRTKVHIGGVLSAHTMETFSRSENETEVKQDVKAGLEGAVKGWGGGATAGHGNTQGTITTSQNRKLNVKYIVNGGDYTKIQNTEEWVASTNQSEHWRVIEVTEVTAVADLLPQPIRGQVKDLLKPLLGKWVDVEKVPGLESLPVSVYRPKGAIPAGWFWLGDTADASKALLVKPTLPARSGRNPALTSLHQGSGMTEQPFVDLPQYQYLSTYFGSFAHDTPPGSTLRGLRPDHVLPGRYEMHGDTISTAVYVTRPVDVPFPEDECFDLKSLVRVKLPGSGNPPKPRSALKKSMVLFDSGEK

SEQ ID NO: 5 - protein, 522 aa, Pleurotus eryngii
MAAVLSRQAATAEAVERFQDSSTSVGLVAGSPSRIRRQADNVVLKSISQAGDTLNDVIQDPTRRNKLINDNNLLKGIIMGRDGPVPSSRELIERPDTLRAIINNRATIETTTVEAEFTETLMESNYNSASVKVSAPFVTANSEYSESSSFKNTETEKSMYTSSRYLFPQGRIDFTMPDSGFDDVIKLSPQFTSGVQAALAKATGTEKREALQDLFLEYGHVFRTKVHIGGVLSAHTMETFSRSENETEVKQDIKAGLEGAVKGWGGGATAGHGNTQGTITTSQNRKLDVKYIVNGGDYTKIQNTEEWVASTNQSEHWRVIEVTEVTAVADLLPQPIRGQVKDLLKPLLGKWVDVEKVPGLESFPVSVYRPKDAIPAGWFWLGDTADASKALLVKPTLPARSGRNPALTSLHESSGMTEQPFVDLPQYQYLSTYFGSFAYDTPPGSTLRGLRPDHILPGRYEMHGDTIGTAVYVTRPVDVPFPEDECFDLKSVVRVKLPGSGNPPKPRWALKKSMVLFDSGEE

(19) United States
(12) Patent Application Publication
(10) Pub. No.: US 2024/0287540 A1
(43) Pub. Date: Aug. 29, 2024

(54) NEW BIO-PESTICIDES FOR CONTROLLING PLANT PESTS

(71) Applicants: KMETIJSKI INŠTITUT SLOVENIJE, Ljubljana (SI); Univerza v Ljubljani, Ljubljana (SI)

(72) Inventors: Anastasija PANEVSKA, Sofija (BG); Jaka RAZINGER, Ljubljana (SI); Kristina SEPCIC, Ljubljana (SI); Peter MACEK, Brezovica (SI); Matej SKOCAJ, Materija (SI); Špela MODIC, Ljubljana (SI); Maruša NOVAK, Krško (SI); Matej BUTALA, Ljubljana (SI); Vesna HODNIK, Žabnica (SI); Maja GRUNDNER, Ljubljana (SI); Tom TURK, Ljubljana-Šmartno (SI)

(73) Assignees: KMETIJSKI INŠTITUT SLOVENIJE, Ljubljana (SI); Univerza v Ljubljani, Ljubljana (SI)

(21) Appl. No.: 18/644,512

(22) Filed: Apr. 24, 2024

Related U.S. Application Data
(63) Continuation-in-part of application No. 16/649,385, filed on Mar. 20, 2020.

(51) Int. Cl. C12N 15/82; C07K 14/37

(52) U.S. Cl. CPC C12N 15/8286; C07K 14/37

(57) ABSTRACT

The present invention relates to cytolytic bi-component protein complexes consisting of a plurality of molecules of a member of the aegerolysin family and a plurality of molecules of a member of the MACPF superfamily, and particularly to their use for controlling a plant pest, such as for controlling Colorado potato beetle (Leptinotarsa decemlineata) or Western corn rootworm (Diabrotica virgifera virgifera). More specifically, the invention relates to transgenic plants forming cytolytic bi-component protein complexes by a plurality of molecules of one of the aegerolysins ostreolysin and pleurotolysin A with a plurality of molecules of pleurotolysin B (PlyB) or similar proteins, which have been transformed into said plants to be toxic for the aforementioned agricultural pest insects.

### CROSS REFERENCE TO RELATED APPLICATIONS

This application is a Continuation-in-Part of U.S. application Ser. No. 16/649,385, filed Mar. 20, 2020, which claims priority to International Application No. PCT/EP2017/074877, filed Sep. 29, 2017, each of which is hereby incorporated herein by reference in its entirety.

### SEQUENCE LISTING

The contents of the electronic sequence listing (ALBG1PUS02CIP_Sequence-Listing.xml; Size: 6573 bytes; and Date of Creation: Apr. 23, 2024) is herein incorporated by reference in its entirety.

### TECHNICAL FIELD OF THE INVENTION

The present invention falls within the scope of plant protection and relates to cytolytic bi-component protein complexes consisting of a plurality of molecules, such as at least 20 molecules of a member of the aegerolysin family and a plurality of molecules, such as at least 10 molecules of a member of the MACPF superfamily, and particularly to their use for controlling a plant pest, such as for controlling Colorado potato beetle (Leptinotarsa decemlineata) or Western corn rootworm (Diabrotica virgifera virgifera).

More specifically, the invention relates to cytolytic bi-component protein complexes formed by a plurality of molecules of one of the aegerolysins ostreolysin A6 (OlyA6), pleurotolysin A2 (PlyA2) and erylysin A (EryA) with a plurality of molecules of pleurotolysin B (PlyB) or similar proteins, which have been shown to be toxic for the aforementioned agricultural pest insects.

### Technical Problem

Synthetic chemical pesticides have been the primary tools used to control Colorado potato beetle (CPB) and Western corn rootworm (WCR). Biological pesticides, such as the insecticidal proteins derived from Bacillus thuringiensis have played an important role as alternative for chemical pesticides. However, due to the constant evolution of resistance to pesticides, there is a continuous need for new bio-pesticides that will target specific molecular targets in pests.

### STATE OF THE ART

CPB and WCR have the biggest economic impact and cause enormous damage to potatoes and maize crops (Alyokhin et al., 2008; Gassmann et al., 2011; Jakka et al., 2016). Current methods for controlling CPB and WCR include chemical pesticides that are facing serious problems, due to constant evolution of pesticide resistance (Meinke et al., 1998; Alyokhin et al., 2008; Pereira et al., 2015; Alyokhin et al., 2015; Jakka et al., 2016). Additional problems are the residues of chemical pesticides in food or feed, environmental concerns (Devine et al., 2007), and human health issues. The search for alternative biopesticides is therefore of the utmost importance.

CPB has been driving the development of the modern insecticidal industry since its early beginnings (Casagrande, 1987). Chemical pesticides, as well as some endotoxins from Bacillus thuringiensis subsp. tenebrionis, are generally in use for CPB control. Despite constant development of new pesticides, the problem due to the developing resistance through different mechanisms remains (Alyokhin et al., 2008).

For WCR control, European and American farmers apply granular insecticides or use insecticide-treated seeds. Foliar insecticides are also occasionally applied against adults (Meissle et al., 2009). However, these practices can cause serious health and environmental problems. Alternatively, WCR is controlled by genetically modified maize that expresses Cry toxins from Bacillus thuringiensis (Bt-maize), or by agronomic practices, such as crop rotation (Meissle et al., 2009). However, WCR has evolved resistance to Bt-maize, and has adapted to crop rotation (Gassman et al., 2012; Chu et al., 2014; Jakka et al., 2016). Biological control options have been recommended for WCR in south-eastern Europe in 1998 (Kuhlmann and Burgt, 1998).

Aegerolysins are low molecular (˜15-20 kDa), acidic, beta-structured proteins, found in several eukaryotic and bacterial taxa (Berne et al., 2009; Novak et al., 2015; Butala et al., 2017). The common feature of aegerolysins is their interaction with specific lipids in biological membranes (Sepčić et al., 2004; Ota et al., 2013; Skočaj et al., 2014; Bhat et al., 2015). Aegerolysins from the fungal genus Pleurotus specifically target ceramide phosphoethanolamines (CPE) (FIG. 1), which are the major membrane sphingolipids of invertebrates (particularly insects and molluscs), but are present only in trace amounts in higher taxa (Crone and Bridges, 1963; Itasaka et al., 1973; Vacaru et al., 2013; Bhat et al., 2015). Moreover, these aegerolysins can function as bi-component lytic complexes in combination with a 59-kDa MACPF (membrane-attack-complex/perforin)-protein targeting cell membranes (Tomita et al., 2004; Shibata et al., 2010; Ota et al., 2013; Lukoyanova et al., 2015). Similar binary and quaternary cytolytic complexes in which aegerolysins are combined with larger, non-aegerolysin protein partner(s) have been found also in bacteria Clostridium bifermentas subsp. malaysia (Quareshi et al., 2014), Bacillus thuringiensis (Masson et al., 2004, Kelker et al., 2014) and Alcaligenes faecalis (Yalpani et al., 2017). These heteromeric bacterial aegerolysin-based cytolytic complexes are being exploited as potent insecticides for specific pests. Cry34Ab1, an aegerolysin protein that belongs to the larger group of insect-specific Cry toxins, and its protein partner Cry35Ab1 are already in use (Bt-maize) as tools for controlling WCR larvae. Cry34Ab1 and its partner specifically bind to (glyco)protein receptors in the membrane of epithelial cells in insects midgut, where the damage occurs (Masson et al., 2004; Kaiser-Alexant et al., 2009; Gassman et al., 2011). However, WCR larvae have recently developed resistance for Bt-maize that produces Cry34Ab1/Cry35Ab1 (Ludwick et al., 2017).

Aegerolysins from fungal genus Pleurotus (OlyA6, PlyA2, EryA) (SEQ ID NOs: 1-3 in FIG. 2) in combination with their specific protein partner, PlyB (SEQ ID NO: 4 in FIG. 2), represent novel promising biopesticides for controlling CPB and WCR. The ability of aegerolysins from the fungal genus Pleurotus to target CPE, and to form transmembrane pores in combination with PlyB, means that they can be used as pest-control agents. Moreover, the chances of evolving resistance to them should be minute, due to the fact that they interact with the membrane lipid receptor, and not with pest proteins that are prone to variation and thus evolvement of resistance to a pesticide.

### SUMMARY OF THE INVENTION

The present invention can be summarized by the following items:
1. Use of a bi-component protein complex consisting of a plurality of molecules, such as at least 20 molecules of a member of the aegerolysin family and a plurality of molecules, such as at least 10 molecules of a member of the MACPF superfamily for controlling a plant pest.
2. The use according to item 1, where the bi-component protein complex consists of 20 to 30 molecules of a member of the aegerolysin family and 10 to 15 molecules of a member of the MACPF superfamily.
3. The use according to item 1 or 2, wherein the ratio of molecules of a member of the aegerolysin family to molecules of a member of the MACPF superfamily is 2:1.
4. The use according to any one of items 1 to 3, wherein the bi-component protein complex consists of 20 molecules of a member of the aegerolysin family and 10 molecules of a member of the MACPF superfamily, or consists of 22 molecules of a member of the aegerolysin family and 11 molecules of a member of the MACPF superfamily, or consists of 24 molecules of a member of the aegerolysin family and 12 molecules of a member of the MACPF superfamily, or consists of 26 molecules of a member of the aegerolysin family and 13 molecules of a member of the MACPF superfamily, or consists of 28 molecules of a member of the aegerolysin family and 14 molecules of a member of the MACPF superfamily, or consists of 30 molecules of a member of the aegerolysin family and 15 molecules of a member of the MACPF superfamily.
5. The use according to any one of items 1 to 4, wherein the member of the aegerolysin family is an aegerolysin derived from a fungus of the genus Pleurotus.
6. The use according to any one of items 1 to 5, wherein the member of the aegerolysin family is selected from the group consisting of ostreolysin, pleurotolysin A and erylysin A.
7. The use according to any one of items 1 to 6, wherein the member of the aegerolysin family is ostreolysin, such as ostreolysin A6.
8. The use according to item 7, wherein the ostreolysin is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 1.
9. The use according to any one of items 1 to 6, wherein the member of the aegerolysin family is pleurotolysin A, such as pleurotolysin A2.
10. The use according to item 9, wherein the pleurotolysin A is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 2.
11. The use according to any one of items 1 to 6, wherein the member of the aegerolysin family is erylysin A.
12. The use according to item 11, wherein the erylysin A is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 3.
13. The use according to any one of items 1 to 12, wherein the member of the MACPF superfamily is a MACPF domain containing protein derived from a fungus of the genus Pleurotus.
14. The use according to any one of items 1 to 13, wherein the member of the MACPF superfamily is pleurotolysin B (PlyB) or erylysin B (Ery B).
15. The use according to any one of items 1 to 13, wherein the member of the MACPF superfamily is pleurotolysin B (PlyB).
16. The use according to item 15, wherein pleurotolysin B is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 4.
17. The use according to any one of items 1 to 13, wherein the member of the MACPF superfamily is erylysin B (Ery B).
18. The use according to item 17, wherein erylysin B (Ery B) is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 5.
19. The use according to any one of items 1 to 18, wherein the plant pest is an insect.
20. The use according to any one of items 1 to 19, wherein the plant pest is a herbivorous insect.
21. The use according to item 19 or 20, wherein the plant pest is a larva of the insect.
22. The use according to item 19 or 20, wherein the plant pest is an imago of the insect.
23. The use according to any one of items 19 to 22, wherein the insect is of the order Coleoptera.
24. The use according to any one of items 19 to 23, wherein the insect is of the family Chrysomelidae.
25. The use according to any one of items 19 to 24, wherein the insect is of the genus Leptinotarsa.
26. The use according to any one of items 19 to 24, wherein the insect is Leptinotarsa decemlineata (Colorado potato beetle).
27. The use according to any one of items 19 to 24, wherein the insect is of the genus Diabrotica.
28. The use according to any one of items 19 to 24, wherein the insect is Diabrotica virgifera virgifera (Western corn rootworm).
29. The use according to any one of items 19 to 24, wherein the insect is selected from the group consisting Leptinotarsa decemlineata (Colorado potato beetle) and Diabrotica virgifera virgifera (Western corn rootworm).
30. The use according to any one of items 1 to 24, wherein the plant pest is selected from Colorado potato beetle and Western corn rootworm.
31. The use according to any one of items 1 to 24, wherein the plant pest is Colorado potato beetle, such as Colorado potato beetle larvae.
32. The use according to any one of items 1 to 24, wherein the plant pest is Western corn rootworm.
33. A method for protecting a plant against a plant pest, comprising the step of: applying a composition comprising a plurality of molecules of a member of the aegerolysin family, a plurality of molecules of a member of the MACPF superfamily and a suitable carrier, such as a buffer solution, to a plant in need thereof.
34. A method for controlling a plant pest, comprising the step of: applying a composition comprising a plurality of molecules of a member of the aegerolysin family, a plurality of molecules of a member of the MACPF superfamily and a suitable carrier, such as a buffer solution, to a plant in need thereof.
35. The method according to item 33 or 34, wherein the member of the aegerolysin family is an aegerolysin derived from a fungus of the genus Pleurotus.
36. The method according to any one of items 33 to 35, wherein the member of the aegerolysin family is selected from the group consisting of ostreolysin, pleurotolysin A and erylysin A.
37. The method according to any one of items 33 to 36, wherein the member of the aegerolysin family is ostreolysin, such as ostreolysin A6.
38. The method according to item 37, wherein the ostreolysin is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 1.
39. The method according to any one of items 33 to 36, wherein the member of the aegerolysin family is pleurotolysin A, such as pleurotolysin A2.
40. The method according to item 39, wherein the pleurotolysin A is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 2.
41. The method according to any one of items 33 to 36, wherein the member of the aegerolysin family is erylysin A.
42. The method according to item 41, wherein the erylysin A is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 3.
43. The method according to any one of items 33 to 42, wherein the member of the MACPF superfamily is a MACPF domain containing protein derived from a fungus of the genus Pleurotus.
44. The method according to any one of items 33 to 43, wherein the member of the MACPF superfamily is pleurotolysin B (PlyB) or erylysin B (Ery B).
45. The method according to any one of items 33 to 43, wherein the member of the MACPF superfamily is pleurotolysin B (PlyB).
46. The method according to item 45, wherein pleurotolysin B is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 4.
47. The method according to any one of items 33 to 43, wherein the member of the MACPF superfamily is erylysin B (Ery B).
48. The use according to item 47, wherein erylysin B (Ery B) is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 5.
49. The method according to any one of items 33 to 48, wherein the molar ratio between the member of the aegerolysin family and the member of the MACPF superfamily is in the range from about 3:1 to about 1000:1, such as about 5:1, about 10:1, about 20:1, about 25:1, about 30:1, about 40:1, about 50:1, about 60:1, about 70:1, about 80:1, about 90:1, about 100:1, about 200:1, about 300:1, about 400:1, about 500:1, about 600:1, about 700:1, about 800:1, or about 900:1, or about 1000:1.
50. The method according to any one of items 33 to 49, wherein the plant pest is an insect.
51. The method according to any one of items 33 to 50, wherein the plant pest is a herbivorous insect.
52. The method according to item 50 or 51, wherein the plant pest is a larva of the insect.
53. The method according to item 50 or 51, wherein the plant pest is an imago of the insect.
54. The method according to any one of items 50 to 53, wherein the insect is of the order Coleoptera.
55. The method according to any one of items 50 to 54, wherein the insect is of the family Chrysomelidae.
56. The method according to any one of items 50 to 55, wherein the insect is of the genus Leptinotarsa.
57. The method according to any one of items 50 to 55, wherein the insect is Leptinotarsa decemlineata (Colorado potato beetle).
58. The method according to any one of items 50 to 55, wherein the insect is of the genus Diabrotica.
59. The method according to any one of items 50 to 55, wherein the insect is Diabrotica virgifera virgifera (Western corn rootworm).
60. The method according to any one of items 50 to 55, wherein the insect is selected from the group consisting Leptinotarsa decemlineata (Colorado potato beetle) and Diabrotica virgifera virgifera (Western corn rootworm).
61. The method according to any one of items 33 to 49, wherein the plant pest is selected from Colorado potato beetle and Western corn rootworm.
62. The method according to any one of items 33 to 49, wherein the plant pest is Colorado potato beetle, such as Colorado potato beetle.
63. The method according to any one of items 33 to 49, wherein the plant pest is Western corn rootworm.
64. Use of a bi-component protein complex as defined in any one of items 1 to 18 or a composition as defined in any one of items 33 to 49 for the preparation of a plant protection agent.
65. A transgenic plant or progeny thereof which expresses or is capable of expressing a bi-component protein complex as defined in any one of items 1 to 18.
66. The transgenic plant or progeny thereof according to item 65, comprising (such as stably transformed with) one or more recombinant nucleic acid molecules comprising nucleotide sequences that encode a bi-component protein complex as defined in any one of items 1 to 18, said nucleotide sequences being operably linked to at least one promoter that is functional in said plant cell to cause the production of mRNA molecules.
67. The transgenic plant or progeny thereof according to item 65 or 66, wherein said transgenic plant or progeny thereof is a crop plant.
68. The transgenic plant or progeny thereof according to any one of items 65 to 67, wherein said transgenic plant or progeny thereof is a potato plant or maize plant.
69. The transgenic plant or progeny thereof according to any one of items 65 to 68, wherein said transgenic plant or progeny thereof is a potato plant.
70. The transgenic plant or progeny thereof according to any one of items 65 to 68, wherein said transgenic plant or progeny thereof is a maize plant.
71. A method for protecting a plant against a plant pest and/or controlling or treating a plant pest, comprising the step of: transforming said plant by introducing one or more nucleic acid construct(s) encoding a bi-component protein complex in a plant genome, consisting of a plurality of molecules selected from the group consisting of ostreolysin and pleurotolysin A and a plurality of molecules of pleurotolysin B (PlyB).
72. The method according to item 71, wherein the transformed plant is subjected to an assay for verifying that said transformed plant has resistance against said plant pest.
73. The method according to item 72, wherein said assay comprises growing the transformed plant and the control plant not being transformed, and then contacting both plants with said plant pest to assess the difference in resistance to said plant pest.
74. The method according to item 72, wherein said assay comprises growing the transformed plants and then contacting half of the plants with said plant pest to assess the difference in resistance to said plant pest.
75. The method according to item 71, wherein the ostreolysin is a polypeptide comprising an amino acid sequence having at least 95% sequence identity with SEQ ID NO: 1.
76. The method according to item 71, wherein the pleurotolysin A is a polypeptide comprising an amino acid sequence having at least 95% sequence identity with SEQ ID NO: 2.
77. The method according to item 71, wherein pleurotolysin B is a polypeptide comprising an amino acid sequence having at least 95% sequence identity with SEQ ID NO: 4.
78. The method according to item 71, wherein the plant pest is an insect of the family Chrysomelidae.
79. The method according to item 71, wherein the plant pest is an insect.
80. The method according to item 71, wherein the plant pest is a larva of the insect or an imago of the insect.
81. The method according to item 79, wherein the insect is of the genus Leptinotarsa.
82. The method according to item 79, wherein the insect is Leptinotarsa decemlineata (Colorado potato beetle).
83. The method according to item 79, wherein the insect is of the genus Diabrotica.
84. The method according to item 81, wherein the insect is Diabrotica virgifera virgifera (Western corn rootworm).
85. The method according to item 79, wherein the insect is selected from the group consisting Leptinotarsa decemlineata (Colorado potato beetle) and Diabrotica virgifera virgifera (Western corn rootworm).
86. The method according to item 71, wherein the plant is a crop plant.
87. The method according to item 71, wherein the plant is a potato plant.
88. The method according to item 71, wherein the plant is a maize plant.
89. A transgenic plant or progeny thereof which expresses or is capable of expressing a bi-component protein complex as defined in any one of items 71 to 88.
90. The transgenic plant or progeny thereof according to item 89, wherein said plant was transformed by introducing one or more nucleic acid construct(s) encoding a bi-component protein complex in a plant genome, consisting of a plurality of molecules selected from the group consisting of ostreolysin and pleurotolysin A and a plurality of molecules of pleurotolysin B (PlyB), so as to obtain said transgenic plant or progeny thereof.

### BRIEF DESCRIPTION OF DRAWINGS

FIG. 1. Structure of ceramide phophoethanolamines (CPE). CPEs are composed of a ceramide residue (long-chain nitrogen base, which is amide-bonded to a 12-24 C atom fatty acid, designated as R2) linked via 1-hydroxy group by a phosphodiester bonding to ethanolamine. The long-chain nitrogen base is either 1,3-dihydroxy C14:1,1,3-dihydroxy C16:1 or 1,3-dihydroxy C18:1, indicated by R1.

FIG. 2. Amino acid sequences of OlyA6 (SEQ ID NO: 1), PlyA2 (SEQ ID NO: 2), EryA (SEQ ID NO: 3), PlyB (SEQ ID NO: 4) and EryB (SEQ ID NO: 5).

FIG. 3. Feeding bioassay on Colorado potato beetle (CPB). Each potato leaf disk was soaked in OlyA6/PlyB, PlyA2/PlyB or EryA/PlyB mixture (0.5 mg/mL for OlyA6, PlyA2 or EryA and 0.04 mg/ml for PlyB) for 5 min and then transferred in each well on the microplate, to which a Colorado potato beetle larva was added. The survival rate was measured daily for 5 days. The weight of the larvae was measured on day 1 and day 5.

FIG. 4. Feeding bioassay on Western corn rootworm. Protein mixtures (PlyA2/PlyB) and artificial diet were mixed (1:1, v:v) and applied in each well. The final protein concentration was 0.5 mg/ml of PlyA2 and 0.04 mg/ml of PlyB. A single Western corn rootworm beetle was transferred in each well and observed for 7 days.

FIG. 5. Comparison of the development between OlyA6/PlyB-treated Colorado potato beetle (CPB) larvae and buffer-treated larvae (day 5). Buffer-treated CPB young larvae showed constant increase in weight and development (A), while CPB larvae treated with OlyA6/PlyB showed no weight change between day 1 and day 5, probably as a result of changed feeding behaviour after day 1 (B).

FIG. 6. Survival rate and weight change of Colorado potato beetle (CPB) during the feeding bioassay. Feeding of CPB larvae with leaf disks treated with OlyA6/PlyB, PlyA2/PlyB or EryA/PlyB protein mixtures (0.5 mg/mL for OlyA6, PlyA2, EryA and 0.04 mg/ml for PlyB) caused significant larval mortality on day 5 after initiation of feeding in the young larval group (L1+L2) (A). Only EryA/PlyB caused significant weight change in the young larval group (B). EryA/PlyB did not cause significant larval mortality in the old larval group (L3+L4) (C), while showing significant weight change (D). OlyA6/PlyB and PlyA2/PlyB caused significant larval mortality (C) in the old group as well as significant weight change (D). In L1 and L2, the pronotum is entirely black. In L3, the anterior margin of the pronotum appears orange-brown. In L4, about half the pronotum is light brown anteriorly. Asterisks indicate significant differences between the tested protein mixtures and buffer, which was used as a negative control (P<0.05).

FIG. 7. Feeding bioassay on Western corn rootworm (WCR). Protein mixtures (OlyA6/PlyB, PlyA2/PlyB or EryA/PlyB) and artificial diet were mixed (1:1, v:v) and applied to each well. The final concentration of OlyA6, EryA or PlyA2 was 0.5 mg/mL and 0.04 mg/ml of PlyB. A single Western corn rootworm beetle was transferred to each well and observed for 7 days. OlyA6/PlyB caused significant mortality of adult WCR, resulting in a median survival time of 5 days. Asterisks indicate significant differences between the tested protein mixtures and buffer, which was used as a negative control (P<0.05).

FIG. 8. The interaction of OlyA6, PlyA2 or EryA, alone or in combination with PlyB, with lipid vesicles composed of CPE:POPC:Chol (5:47.5:47.5, mol:mol:mol). OlyA6 and PlyA2 were injected in concentration of 0.25 μM and EryA was injected in concentration of 5 μM. PlyB was injected in concentration 20 nM when combined with OlyA6 and PlyA2, and 0.4 μM when combined with EryA. OlyA6 (A), PlyA2 (B) and EryA (C) specifically interact with membranes containing CPE and their interaction is stabilized in the presence of PlyB. CPE, ceramide phosphoethanolamine; POPC, palmitoyl-2-oleoyl-sn-glycero-3-phosphocholine; Chol, cholesterol.

FIG. 9. Lytic activity of OlyA6/PlyB, PlyA2/PlyB and EryA/PlyB. Tested protein mixtures OlyA6/PlyB and PlyA2/PlyB (10 μg/mL for OlyA6, PlyA2 or EryA and 0.8 μg/mL for PlyB) show permeabilization of artificial lipid vesicles containing CPE (A) as well as vesicles composed of SM:Chol (1:1, mol:mol) (B), while EryA/PlyB (10 μg/mL for EryA and 0.8 μg/mL for PlyB) is lytic only for vesicles containing CPE. CPE, ceramide phosphoethanolamine; POPC, palmitoyl-2-oleoyl-sn-glycero-3-phosphocholine; Chol, cholesterol; SM, sphingomyelin.

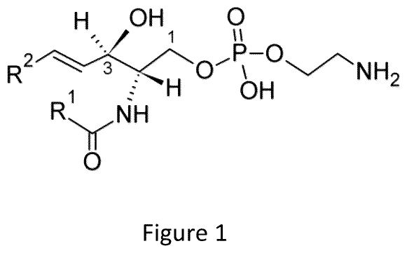

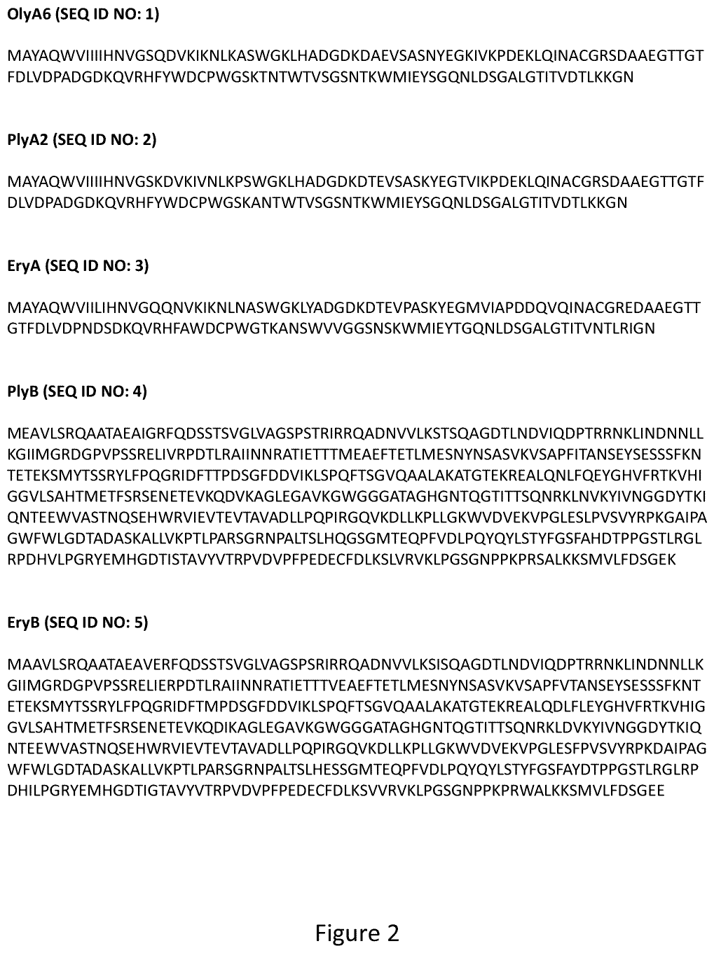

### DETAILED DESCRIPTION OF THE INVENTION

The present invention relates to cytolytic bi-component protein complexes consisting of a plurality of molecules, such as at least 20 molecules of a member of the aegerolysin family and a plurality of molecules, such as at least 10 molecules of a member of the MACPF superfamily, and particularly to their use for controlling a plant pest, such as for controlling Colorado potato beetle (Leptinotarsa decemlineata) or Western corn rootworm (Diabrotica virgifera virgifera). The present invention thus provides cytolytic bi-component protein complexes consisting of a plurality of molecules of a member of the aegerolysin family and a plurality of molecules of a member of the MACPF superfamily which are useful as pesticides.

As described in the Examples, cytolytic bi-component protein complexes composed of molecules of a member of the aegerolysin family and molecules of a member of the MACPF superfamily show toxic effect when ingested by invertebrates, particularly insects. These results indicate that the bi-component protein complexes damage the gut membranes. The subject of the invention thus indicates that bi-component protein complexes consisting of a plurality of molecules of a member of the aegerolysin family, such as OlyA6, PlyA2 or EryA, and a plurality of molecules of a member of the MACPF superfamily, such as PlyB, can serve as alternative biopesticides that can reduce the risk to the environment and to human health.

According to one aspect, the present invention provides the use of a bi-component protein complex consisting of a plurality of molecules of a member of the aegerolysin family and a plurality of molecules of a member of the MACPF superfamily for controlling a plant pest.

With »plurality of molecules« it is meant any number of molecules of a member of the aegerolysin family and any number of molecules of a member of the MACPF superfamily which allows the formation of transmembrane pores.

The bi-component protein complex may, for example, consist of 20 to 30 molecules of a member of the aegerolysin family and 10 to 15 molecules of a member of the MACPF superfamily.

Thus, according to certain embodiments, the bi-component protein complex consists of 20 to 30 molecules of a member of the aegerolysin family and 10 to 15 molecules of a member of the MACPF superfamily. According to certain embodiments, the bi-component protein complex consists of 22 to 30 molecules of a member of the aegerolysin family and 11 to 15 molecules of a member of the MACPF superfamily. According to certain embodiments, the bi-component protein complex consists of 24 to 30 molecules of a member of the aegerolysin family and 12 to 15 molecules of a member of the MACPF superfamily. According to certain embodiments, the bi-component protein complex consists of 26 to 30 molecules of a member of the aegerolysin family and 13 to 15 molecules of a member of the MACPF superfamily. According to certain embodiments, the bi-component protein complex consists of 28 to 30 molecules of a member of the aegerolysin family and 14 to 15 molecules of a member of the MACPF superfamily. According to certain embodiments, the bi-component protein complex consists of 22 to 28 molecules of a member of the aegerolysin family and 11 to 14 molecules of a member of the MACPF superfamily.

Generally, the bi-component protein complex is formed by a plurality of molecules of a member of the aegerolysin family and a plurality of molecules of a member of the MACPF superfamily in a ratio 2:1. In other words, the ratio of molecules of a member of the aegerolysin family to molecules of a member of the MACPF superfamily is 2:1.

According to some embodiments, the bi-component protein complex consists of 20 molecules of a member of the aegerolysin family and 10 molecules of a member of the MACPF superfamily, or consists of 22 molecules of a member of the aegerolysin family and 11 molecules of a member of the MACPF superfamily, or consists of 24 molecules of a member of the aegerolysin family and 12 molecules of a member of the MACPF superfamily, or consists of 26 molecules of a member of the aegerolysin family and 13 molecules of a member of the MACPF superfamily, or consists of 28 molecules of a member of the aegerolysin family and 14 molecules of a member of the MACPF superfamily, or consists of 30 molecules of a member of the aegerolysin family and 15 molecules of a member of the MACPF superfamily.

According to particular embodiments, the bi-component protein complex consists of 26 molecules of a member of the aegerolysin family and 13 molecules of a member of the MACPF superfamily.

Since the bi-component protein complex can be (or is) formed in situ it will be understood that the molecular composition and frequency may vary, meaning that different bi-component protein complexes formed of the same components may be present on the plane of the lipid bilayer varying in their molecular composition and frequency. As a non-limting example, Lukoyanova et al. (2015) and Ota et al. (2013), dealing with structures of PlyA/PlyB and OlyA6/PlyB based complexes, have shown that the pores formed by aegerolysins and PlyB in situ usually come in the following molecular compositions (the % denotes the frequency of different molecular composition on the plane of the lipid bilayer): 26 PlyA:13 PlyB (75%), 24 PlyA:12 PlyB (15%), 22 PlyA:11 PlyB (5%), and 28 PlyA:14 PlyB (5%). Hence, the bi-component protein complex may be a mixture of component protein complexes formed by the same components varying in their molecular composition.

Members of the aegerolysin family as well as members of the MACPF superfamily from a range of fungi and bacteria have been described in the scientific literature (e.g. Ota et al., 2014; Butala et al., 2017; Anderluh et al., 2014). The aegerolysin family is a family of proteins which are characterized in that they contain an aegerolysin domain. The MACPF superfamily is a family of proteins which are characterized in that they contain a MACPF (Membrane Attack Complex/Perforin) domain. Members of the aegerolysin family specifically target ceramide phosphoethanolamines, which are major membrane sphingolipids of invertebrates (particular insects and molluscs). Some members of the MACPF superfamily have cytolytic activity, and those are useful according to the present invention.

Members of the aegerolysin family as well as members of the MACPF superfamily for use according to the present invention may be of fungal or bacterial origin. Non-limiting examples of members of the aegerolysin family include aegerolysins derived from a fungus of the genus Pleurotus, such as from Pleurotus ostreatus or Pleurotus eryngii. Non-limiting examples of members of the MACPF superfamily include MACPF-containing proteins derived from a fungus of the genus Pleurotus, such as from Pleurotus ostreatus or Pleurotus eryngii.

Therefore, according to certain embodiments, the member of the aegerolysin family is an aegerolysin derived from a fungus of the genus Pleurotus.

According to some embodiments, the member of the aegerolysin family is an aegerolysin derived from the fungus Pleurotus ostreatus. According to some other embodiments, the member of the aegerolysin family is an aegerolysin derived from the fungus Pleurotus eryngii.

According to certain embodiments, the member of the MACPF superfamily is a MACPF-containing protein derived from a fungus of the genus Pleurotus.

According to some embodiments, the member of the MACPF superfamily is a MACPF-containing protein derived from the fungus Pleurotus ostreatus. According to some other embodiments, the member of the MACPF superfamily is a MACPF-containing protein derived from the fungus Pleurotus eryngii.

Non-limiting examples of aegerolysins include ostreolysin, pleurotolysin A and erylysin A.

According to certain embodiments, the member of the aegerolysin family is selected from the group consisting of ostreolysin, pleurotolysin A and erylysin A.

According to some embodiments, the member of the aegerolysin family is ostreolysin, such as ostreolysin A6. According to some specific embodiments, the ostreolysin is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 1. According to some specific embodiments, the ostreolysin is a polypeptide comprising an amino acid sequence having at least 55% sequence identity with SEQ ID NO: 1. According to some specific embodiments, the ostreolysin is a polypeptide comprising an amino acid sequence having at least 60% sequence identity with SEQ ID NO: 1. According to some specific embodiments, the ostreolysin is a polypeptide comprising an amino acid sequence having at least 65% sequence identity with SEQ ID NO: 1.

According to some specific embodiments, the ostreolysin is a polypeptide comprising an amino acid sequence having at least 70% sequence identity with SEQ ID NO: 1. According to some specific embodiments, the ostreolysin is a polypeptide comprising an amino acid sequence having at least 75% sequence identity with SEQ ID NO: 1. According to some specific embodiments, the ostreolysin is a polypeptide comprising an amino acid sequence having at least 80% sequence identity with SEQ ID NO: 1. According to some specific embodiments, the ostreolysin is a polypeptide comprising an amino acid sequence having at least 85% sequence identity with SEQ ID NO: 1. According to some specific embodiments, the ostreolysin is a polypeptide comprising an amino acid sequence having at least 90% sequence identity with SEQ ID NO: 1. According to some specific embodiments, the ostreolysin is a polypeptide comprising an amino acid sequence having at least 95% sequence identity with SEQ ID NO: 1. According to some specific embodiments, the ostreolysin is a polypeptide comprising an amino acid sequence having at least 97% sequence identity with SEQ ID NO: 1. According to some specific embodiments, the ostreolysin is a polypeptide comprising an amino acid sequence having at least 99% sequence identity with SEQ ID NO: 1. Such polypeptide(s) suitably has the same or similar property than the reference polypeptide of SEQ ID NO: 1, i.e. binding to CPEs and forming a cytolytic complex with pleurotolysin B and similar proteins. The property can be determined in accordance with the following membrane permeabilization test:

### Membrane Permeabilization Test

### 1. Principle:

Calcein-loaded small unilamellar vesicles (SUVs) containing CPE are used in order to confirm the lytic activity of a polypeptide to be tested (i.e. a polypeptide having the indicated % sequence identity to an aegerolysin, such as ostreolysin of SEQ ID NO: 1-herein after »test polypeptide«) with the protein partner PlyB (SEQ ID NO: 4).

To prepare the SUVs, lipid films with defined molar proportions of lipids (CPE:POPC:Chol [5:47.5:47.5, mol:mol:mol]) are prepared by removing the organic solvent from lipid solutions by rotary evaporation and vacuum drying. Lipids, at final concentration of 5 mg/mL, are swollen in 80 mM calcein and vortexed vigorously to give multilamellar liposomes (MLVs). The suspension of MLVs is sonicated for 15 minutes on ice with 10 sec on/off cycles to prepare SUVs. Extra-vesicular calcein is removed from SUV suspension by gel filtration on a Sephadex G-50 (medium) column, where vesicle buffer composed of 140 mM NaCl, 20 mM TRIS·HCl, pH 8.0 is used as a mobile phase. The lytic activity of the protein complex is assayed using a fluorescence microplate reader at 25° C.

### 2. Procedure:

Protein mixtures comprising the test polypeptide and PlyB (12.5:1, mol:mol) are dispensed into a multi-well microplate at the following concentrations: 10 μg/mL test polypeptide combined with 0.8 μg/ml PlyB. The final volume of the proteins in each well of the microtiter plate, diluted in vesicle buffer (140 mM NaCl, 20 mM TRIS·HCl, pH 8.0), is 100 μL. Calcein-loaded SUVs (5 μg/mL) are added to the protein mixtures. The SUVs are excited at 485 nm and the intensity of the emitted fluorescence of released calcein is monitored at 535 nm for 30 min with 20 s intervals. The intensity of the emitted fluorescence at t=30 is determined as F. SUVs are fully lysed with 1 mM Triton X-100 (positive control), where Fₘₐₓ is determined as a maximal fluorescence intensity following the lysis of all the SUVs. F₀ is the fluorescence of SUVs at t=30 in the absence of lytic protein mixtures, or in the absence of Triton X-100. The percentage of calcein release R (%) is calculated as:

$$R ( \% ) = \frac{F - F o}{F max - F o} * 100$$

The protein complex can be considered as lytic when the R value is ≥5% of the positive control.

According to some embodiments, the member of the aegerolysin family is pleurotolysin A, such as pleurotolysin A2. According to some specific embodiments, the pleurotolysin A is a polypeptide comprising an amino acid sequence having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 2. According to some specific embodiments, the pleurotolysin A is a polypeptide comprising an amino acid sequence having at least 55% sequence identity with SEQ ID NO: 2. According to some specific embodiments, the pleurotolysin A is a polypeptide comprising an amino acid sequence having at least 60% sequence identity with SEQ ID NO: 2. According to some specific embodiments, the pleurotolysin A is a polypeptide comprising an amino acid sequence having at least 65% sequence identity with SEQ ID NO: 2. According to some specific embodiments, the pleurotolysin A is a polypeptide comprising an amino acid sequence having at least 70% sequence identity with SEQ ID NO: 2. According to some specific embodiments, the pleurotolysin A is a polypeptide comprising an amino acid sequence having at least 75% sequence identity with SEQ ID NO: 2. According to some specific embodiments, the pleurotolysin A is a polypeptide comprising an amino acid sequence having at least 80% sequence identity with SEQ ID NO: 2. According to some specific embodiments, the pleurotolysin A is a polypeptide comprising an amino acid sequence having at least 85 sequence identity with SEQ ID NO: 2. According to some specific embodiments, the pleurotolysin A is a polypeptide comprising an amino acid sequence having at least 90% sequence identity with SEQ ID NO: 2. According to some specific embodiments, the pleurotolysin A is a polypeptide comprising an amino acid sequence having at least 95% sequence identity with SEQ ID NO: 2. According to some specific embodiments, the pleurotolysin A is a polypeptide comprising an amino acid sequence having at least 97% sequence identity with SEQ ID NO: 2. According to some specific embodiments, the pleurotolysin A is a polypeptide comprising an amino acid sequence having at least 99% sequence identity with SEQ ID NO: 2. Such polypeptide(s) suitably has the same or similar property than the reference polypeptide of SEQ ID NO: 2, i.e. binding to CPEs and forming a cytolytic complex with pleurotolysin B and similar proteins. The property can be determined in accordance with the membrane permeabilization test described above.

According to some embodiments, the member of the aegerolysin family is erylysin A. According to some specific embodiments, the erylysin A is a polypeptide having at least 50%, such as at least 55%, at least 60%, at least 65%, at least 70%, at least 75%, at least 80%, at least 85%, at least 90%, at least 95%, at least 97% or at least 99% sequence identity with SEQ ID NO: 3. According to some specific embodiments, the erylysin A is a polypeptide having at least 55% sequence identity with SEQ ID NO: 3. According to some specific embodiments, the erylysin A is a polypeptide having at least 60% sequence identity with SEQ ID NO: 3. According to some specific embodiments, the erylysin A is a polypeptide having at least 65% sequence identity with SEQ ID NO: 3. According to some specific embodiments, the erylysin A is a polypeptide having at least 70% sequence identity with SEQ ID NO: 3. According to some specific embodiments, the erylysin A is a polypeptide having at least 75% sequence identity with SEQ ID NO: 3. According to some specific embodiments, the erylysin A is a polypeptide having at least 80% sequence identity with SEQ ID NO: 3. According to some specific embodiments, the erylysin A is a polypeptide having at least 85% sequence identity with SEQ ID NO: 3. According to some specific embodiments, the erylysin A is a polypeptide having at least 90% sequence identity with SEQ ID NO: 3. According to some specific embodiments, the erylysin A is a polypeptide having at least 95% sequence identity with SEQ ID NO: 3. According to some specific embodiments, the erylysin A is a polypeptide comprising an amino acid sequence having at least 97% sequence identity with SEQ ID NO: 3. According to some specific embodiments, the erylysin A is a polypeptide comprising an amino acid sequence having at least 99% sequence identity with SEQ ID NO: 3. Such polypeptide(s) suitably has the same or similar property than the reference polypeptide of SEQ ID NO: 3, i.e. binding to CPEs and forming a cytolytic complex with pleurotolysin B and similar proteins. The property can be determined in accordance with the membrane permeabilization test described above.

Non-limiting examples of a member of the MACPF superfamily include pleurotolysin B (PlyB), erylysin B (Ery B) or a similar protein from the fungi Sphaerobolus stellatus, Moniliophtora perniciosa, Trametes pubescens, and Heterobasidion irregulare.

According to certain embodiments, the member of the MACPF superfamily is pleurotolysin B (PlyB) or erylysin B (Ery B).

According to some embodiments, the member of the MACPF superfamily is pleurotolysin B (PlyB). According to some specific embodiments, pleurotolysin B is a polypeptide comprising an amino acid sequence having at least 50%, such as at least 55%, at least 60%, at least 65%, at least 70%, at least 75% %, at least 80%, at least 85%, at least 90%, at least 95%, at least 97% or at least 99% sequence identity with SEQ ID NO: 4. According to some specific embodiments, pleurotolysin B is a polypeptide comprising an amino acid sequence having at least 55% sequence identity with SEQ ID NO: 4. According to some specific embodiments, pleurotolysin B is a polypeptide comprising an amino acid sequence having at least 60% sequence identity with SEQ ID NO: 4. According to some specific embodiments, pleurotolysin B is a polypeptide comprising an amino acid sequence having at least 65% sequence identity with SEQ ID NO: 4. According to some specific embodiments, pleurotolysin B is a polypeptide comprising an amino acid sequence having at least 70% sequence identity with SEQ ID NO: 4. According to some specific embodiments, pleurotolysin B is a polypeptide comprising an amino acid sequence having at least 75% % sequence identity with SEQ ID NO: 4. According to some specific embodiments, pleurotolysin B is a polypeptide comprising an amino acid sequence having at least 80% sequence identity with SEQ ID NO: 4. According to some specific embodiments, pleurotolysin B is a polypeptide comprising an amino acid sequence having at least 85% sequence identity with SEQ ID NO: 4. According to some specific embodiments, pleurotolysin B is a polypeptide comprising an amino acid sequence having at least 90% sequence identity with SEQ ID NO: 4. According to some specific embodiments, pleurotolysin B is a polypeptide comprising an amino acid sequence having at least 95% sequence identity with SEQ ID NO: 4. According to some specific embodiments, pleurotolysin B is a polypeptide comprising an amino acid sequence having at least at least 97% sequence identity with SEQ ID NO: 4. According to some specific embodiments, pleurotolysin B is a polypeptide comprising an amino acid sequence having at least 99% sequence identity with SEQ ID NO: 4. Such polypeptide(s) suitably has the same or similar cytolytic activity with ostreolysin, pleurotolysin A, erylysin A or a similar protein than the reference polypeptide of SEQ ID NO: 4. Particularly, such polypeptide(s) contains a functional MACPF domain. The cytolytic activity can be determined by suitable tests available to the skilled person, such as by hemolysis assay or by monitoring the calcein release from liposomes. The cytolytic activity may, for example, be determined in accordance with the following membrane permeabilization test:

### Membrane Permeabilization Test

### 1. Principle:

Calcein-loaded small unilamellar vesicles (SUVs) containing CPE are used in order to confirm the lytic activity of polypeptide to be tested (i.e. a polypeptide having the indicated % sequence idenity to a member of the MACPF superfamily, such as pleurotolysin B of SEQ ID NO: 4—herein after »test polypeptide«) with one of the protein partners ostreolysin (SEQ ID NO: 1), pleurotolysin A (SEQ ID NO: 2) or erylysin A (SEQ ID NO: 3).

To prepare the SUVs, lipid films with defined molar proportions of lipids (CPE:POPC:Chol [5:47.5:47.5, mol:mol:mol]) are prepared by removing the organic solvent from lipid solutions by rotary evaporation and vacuum drying. Lipids, at final concentration of 5 mg/mL, are swollen in 80 mM calcein and vortexed vigorously to give multilamellar liposomes (MLVs). The suspension of MLVs is sonicated for 15 minutes on ice with 10 sec on/off cycles to prepare SUVs. Extra-vesicular calcein is removed from SUV suspension by gel filtration on a Sephadex G-50 (medium) column, where vesicle buffer composed of 140 mM NaCl, 20 mM TRIS·HCl, pH 8.0 is used as a mobile phase. The lytic activity of the protein complex is assayed using a fluorescence microplate reader at 25° C.

### 2. Procedure:

Protein mixtures comprising the test polypeptide and ostreolysin, pleurotolysin A or erylysin A (1:12.5, mol:mol) are dispensed into a multi-well microplate at the following concentrations: 10 μg/ml test aegerolysin combined with 0.8 μg/mL PlyB. The final volume of the proteins in each well of the microtiter plate, diluted in vesicle buffer (140 mM NaCl, 20 mM TRIS·HCl, pH 8.0), is 100 μL. Calcein-loaded SUVs (5 μg/mL) are added to the protein mixtures. The SUVs are excited at 485 nm and the intensity of the emitted fluorescence of released calcein is monitored at 535 nm for 30 min with 20 s intervals. The intensity of the emitted fluorescence at t=30 is determined F. SUVs are fully lysed with 1 mM Triton X-100 (positive control), where Fₘₐₓ is determined as a maximal fluorescence intensity following the lysis of all the SUVs. F₀ is the fluorescence of SUVs at t=30 in the absence of lytic protein mixtures, or in the absence of Triton X-100. The percentage of calcein release R (%) is calculated as:

$$R ( \% ) = \frac{F - F o}{F max - F o} * 100$$

The protein complex can be considered as lytic when the R value is >5% of the positive control.

According to some embodiments, the member of the MACPF superfamily is erylysin B (Ery B). According to some specific embodiments, erylysin B (Ery B) is a polypeptide comprising an amino acid sequence having at least 50%, such as at least 55%, at least 60%, at least 65%, at least 75% %, at least 80%, at least 85%, at least 90%, at least 95%, at least 97% or at least 99% sequence identity with SEQ ID NO: 5. According to some specific embodiments, erylysin B (Ery B) is a polypeptide comprising an amino acid sequence having at least 55% sequence identity with SEQ ID NO: 5. According to some specific embodiments, erylysin B (Ery B) is a polypeptide comprising an amino acid sequence having at least 60% sequence identity with SEQ ID NO: 5. According to some specific embodiments, erylysin B (Ery B) is a polypeptide comprising an amino acid sequence having at least 65% sequence identity with SEQ ID NO: 5. According to some specific embodiments, erylysin B (Ery B) is a polypeptide comprising an amino acid sequence having at least 70% sequence identity with SEQ ID NO: 5. According to some specific embodiments, erylysin B (Ery B) is a polypeptide comprising an amino acid sequence having at least 75% % sequence identity with SEQ ID NO: 5. According to some specific embodiments, erylysin B (Ery B) is a polypeptide comprising an amino acid sequence having at least 80% sequence identity with SEQ ID NO: 5. According to some specific embodiments, erylysin B (Ery B) is a polypeptide comprising an amino acid sequence having at least 85% sequence identity with SEQ ID NO: 5. According to some specific embodiments, erylysin B (Ery B) is a polypeptide comprising an amino acid sequence having at least 90% sequence identity with SEQ ID NO: 5. According to some specific embodiments, erylysin B (Ery B) is a polypeptide comprising an amino acid sequence having at least 95% sequence identity with SEQ ID NO: 5. According to some specific embodiments, erylysin B (Ery B) is a polypeptide comprising an amino acid sequence having at least 97% sequence identity with SEQ ID NO: 5. According to some specific embodiments, erylysin B (Ery B) is a polypeptide comprising an amino acid sequence having at least 99% sequence identity with SEQ ID NO: 5. Such polypeptide(s) suitably has the same or similar cytolytic activity with ostreolysin, pleurotolysin A, erylysin A or a similar protein than the reference polypeptide of SEQ ID NO: 5. Particularly, such polypeptide(s) contains a functional MACPF domain. The cytolytic activity can be determined by suitable tests available to the skilled person, such as by hemolysis assay or by monitoring the calcein release from liposomes. The cytolytic activity may, for example, be determined in accordance with the membrane permeabilization test described above.

According to certain embodiments, the member of the aegerolysin family is ostreolysin, such as ostreolysin A6, and the member of the MACPF superfamily is pleurotolysin B.

According to certain embodiments, the member of the aegerolysin family is pleurotolysin A, such as pleurotolysin A2, and the member of the MACPF superfamily is pleurotolysin B.

According to certain embodiments, the member of the aegerolysin family is erylysin A and the member of the MACPF superfamily is pleurotolysin B.

According to certain embodiments, the member of the aegerolysin family is ostreolysin, such as ostreolysin A6, and the member of the MACPF superfamily is erylysin B.

According to certain embodiments, the member of the aegerolysin family is pleurotolysin A, such as pleurotolysin A2, and the member of the MACPF superfamily is erylysin B.

According to certain embodiments, the member of the aegerolysin family is erylysin A and the member of the MACPF superfamily is erylysin B.

The bi-component protein complex may generally be used at a concentration ranging from about 1.5 μg/ml (0.1 μM of a member of the aegerolysin family in a 1000:1 molar ratio with a member of the MACPF superfamily) to 34.5 mg/ml (1 mM of a member of the aegerolysin family in a 3:1 molar ratio with a member of the MACPF superfamily). That is the concentration of the member of the aegerolysin family used is usually in the range of 0.1 μM to about 1 mM, such as from about 0.5 μM to about 1 mM, about 1 μM to about 1 mM, about 10 μM to about 1 mM, about 50 μM to about 1 mM, about 100 μM to about 1 mM, or about 500 μM to about 1 mM, about 0.1 μM to 500 μM, 0.5 μM to about 500 μM, about 1 μM to about 500 μM, about 10 μM to about 500 μM, about 50 μM to about 500 μM, about 100 μM to about 500 μM, 0.5 μM to about 100 μM, about 1 μM to about 100 μM, about 10 μM to about 100 μM, or about 50 μM to about 100 μM. The member of the MACPF superfamily is used in appropriate amounts to obtain the desired molar ratio which is generally in the range from about 3:1 to about 1000:1.

The plant pest to be controlled by using a bi-component protein complex in accordance with the invention can be an insect, such as a herbivorous insect. Therefore, according to certain embodiments, the plant pest is an insect. According to some embodiments, the plant pest is a herbivorous insect.

The plant pest may be a larva and/or an imago of the insect. According to certain embodiments, the plant pest is a larva of the insect. According to certain embodiments, the plant pest is an imago of the insect.

The larva may be in any stage of larval development. According to certain embodiments, the larva is in a larval stage selected from the group consisting of L1, L2, L3, L4, and L5. According to certain embodiments, the larva is in a larval stage selected selected from L1, L2, L3, and L4. According to certain embodiments, the larva is in a larval stage selected selected from L1, L2, and L3. According to certain embodiments, the larva is in a larval stage selected selected from L1 and L2. According to certain embodiments, the larva is in a larval stage selected from the group consisting of L2, L3, L4, and L5. According to certain embodiments, the larva is in a larval stage selected from the group consisting of L2, L3 and L4. According to certain embodiments, the larva is in a larval stage selected from the group consisting of L2 and L3. According to certain embodiments, the larva is in a larval stage selected from the group consisting of L3 and L4. According to some embodiments, the larva is in larval stage L1. According to some embodiments, the larva is in larval stage L2. According to some embodiments, the larva is in larval stage L3. According to some embodiments, the larva is in larval stage L4. According to some embodiments, the larva is in larval stage L5.

According to certain embodiments, the insect is of the order Coleoptera.

According to some embodiments, the insect is of the family Chrysomelidae.

According to some particular embodiments, the insect is of the genus Leptinotarsa.

According to some specific embodiments, the insect is Leptinotarsa decemlineata (Colorado potato beetle).

According to some particular embodiments, the insect is of the genus Diabrotica.

According to some specific embodiments, the insect is Diabrotica virgifera virgifera (Western corn rootworm).

According to some particular embodiments, the insect is of the genus Phyllotreta.

According to some specific embodiments, the insect is Phyllotreta spp.

According to some specific embodiments, the insect is Phyllotreta cruciferae.

According to some specific embodiments, the insect is Phyllotreta striolata.

According to some specific embodiments, the insect is selected from the group consisting Leptinotarsa decemlineata (Colorado potato beetle) and Diabrotica virgifera virgifera (Western corn rootworm).

According to some particular embodiments, the insect is of the genus Lilioceris.

According to some specific embodiments, the insect is Lilioceris merdigera.

According to some specific embodiments, the insect is Lilioceris lilii.

According to some particular embodiments, the insect is of the genus Crioceris.

According to some specific embodiments, the insect is Crioceris duodecimpunctata.

According to some embodiments, the insect is of the family Scarabeidae.

According to some particular embodiments, the insect is of the genus Melolontha.

According to some specific embodiments, the insect is Melolontha melolontha.

According to some particular embodiments, the insect is of the genus Popillia.

According to some specific embodiments, the insect is Popillia japonica (Japanese beetle).

According to some embodiments, the insect is of the family Elateridae.

According to some particular embodiments, the insect is of the genus Agriotes.

According to some specific embodiments, the insect is Agriotes spp.

According to some specific embodiments, the insect is Agriotes lineatus.

According to some specific embodiments, the insect is Agriotes obscurus.

According to some specific embodiments, the insect is Agriotes ustulatus.

According to some specific embodiments, the insect is Agriotes sputator.

According to some embodiments, the insect is of the family Byturidae.

According to some particular embodiments, the insect is of the genus Byturus.

According to some specific embodiments, the insect is Byturus tomentosus.

According to certain embodiments, the plant pest is a coleopteran insect pest.

According to particular embodiments, the plant pest is selected from Colorado potato beetle, Western corn rootworm and other coleopteran insect pests.

According to some embodiments, the plant pest is a Colorado potato beetle.

According to some embodiments, the plant pest is Western corn rootworm.

According to some embodiments, the plant pest is cabbage flea beetle.

According to some embodiments, the plant pest is Japanese beetle.

According to some embodiments, the plant pest is May beetle.

According to some embodiments, the plant pest is Wireworm.

According to some embodiments, the plant pest is Raspberry beetle.

The present invention further provides a method for protecting a plant against a plant pest, comprising the step of: applying a composition comprising a plurality of molecules of a member of the aegerolysin family, a plurality of molecules of a member of the MACPF superfamily and a suitable carrier, such as a buffer solution, to a plant in need thereof.

The present invention further provides a method for controlling a plant pest, comprising the step of: applying a composition comprising a plurality of molecules of a member of the aegerolysin family, a plurality of molecules of a member of the MACPF superfamily and a suitable carrier, such as a buffer solution, to a plant in need thereof.

Generally, the member of the aegerolysin family and the member of the MACPF superfamily are present in the composition in a free, non-complexed form. Once the composition is applied to a plant of interest and molecules of the member of the aegerolysin family and molecules of the member of the MACPF superfamily are ingested by the insect, bi-component protein complexes as described herein are formed in situ on the plasmalemma of epithelial cells of the midgut, leading to the perforation of the gut, and subsequently to the death of the insect.

It is understood that all details provided herein with respect to the bi-component protein complexes, particularly with respect to the member of the aegerolysin family and the member of the MACPF superfamily, and the plant pest, including all embodiments, apply mutatis mutandis to the methods of the present invention.

Generally, the composition can be applied to a plant in need thereof in any suitable dose, frequency and method of administration.

The composition may suitably be in liquid form, and may be applied by spraying, drenching or dropping onto the plant. According to certain embodiments, the composition is applied by drenching. According to certain embodiments, the bi-composition is applied by spraying. According to certain embodiments, the composition is applied by dropping.

Suitably, the carrier is a liquid carrier, and more particularly an aqueous carrier, and the member of the aegerolysin family and the member of the MACPF superfamily are dissolved therein. Suitable carriers are well known to the skilled person. A suitable aqueous carrier may for example be a buffer solution, and more particularly a physiological buffer solution. Suitable buffer systems are well known to the skilled person and include as non-limiting examples Tris, TABS, Bicine, Tricine, HEPES, TES, MOPS and PIPES. The pH of the buffer solution is usually in the range of about 6.5 to about 9, such as in the range from about 7.5 to about 8.5, such as about 8. The buffer solution may comprise further additives such as NaCl and/or glycerol. A non-limiting example of a buffer solution useful according to the present invention is a buffer solution comprising 20-50 mM Tris, 0-200 mM NaCl and 0-1% glycerol in deionized water. A more specific non-limiting example of a buffer solution useful according to the present invention is 20 mM Tris, 0.5% glycerol, pH 8.0, in deionized water.

The concentration of the member of the aegerolysin family used may generally be in the range of 0.1 μM to about 1 mM, such as from about 0.5 μM to about 1 mM, about 1 μM to about 1 mM, about 10 M to about 1 mM, about 50 μM to about 1 mM, about 100 μM to about 1 mM, or about 500 μM to about 1 mM, about 0.1 μM to 500 μM, 0.5 μM to about 500 μM, about 1 μM to about 500 μM, about 10 UM to about 500 μM, about 50 μM to about 500 μM, about 100 μM to about 500 μM, 0.5 μM to about 100 μM, about 1 μM to about 100 μM, about 10 μM to about 100 μM, or about 50 μM to about 100 μM. The member of the MACPF superfamily is used in appropriate amounts to obtain the desired molar ratio in the range from about 3:1 to about 1000:1.

The molar ratio between a member of the aegerolysin family and a member of the MACPF superfamily may generally be in the range from about 3:1 to about 1000:1, such as about 5:1, about 10:1, about 20:1, about 25:1, about 30:1, about 40:1, about 50:1, about 60:1, about 70:1, about 80:1, about 90:1, about 100:1, about 200:1, about 300:1, about 400:1, about 500:1, about 600:1, about 700:1, about 800:1, about 900:1, or about 1000:1.

According to certain embodiments, the molar ratio between a member of the aegerolysin family and a member of the MACPF superfamily is in the range from about 3:1 to about 1000:1. According to some embodiments, the molar ratio between a member of the aegerolysin family and a member of the MACPF superfamily is about 3:1. According to some embodiments, the molar ratio between a member of the aegerolysin family and a member of the MACPF superfamily is about 5:1. According to some embodiments, the molar ratio between a member of the aegerolysin family and a member of the MACPF superfamily is about 10:1. According to some embodiments, the molar ratio between a member of the aegerolysin family and a member of the MACPF superfamily is about 20:1. According to some embodiments, the molar ratio between a member of the aegerolysin family and a member of the MACPF superfamily is about 25:1. According to some embodiments, the molar ratio between a member of the aegerolysin family and a member of the MACPF superfamily is about 30:1. According to some embodiments, the molar ratio between a member of the aegerolysin family and a member of the MACPF superfamily is about 40:1. According to some embodiments, the molar ratio between a member of the aegerolysin family and a member of the MACPF superfamily is about 50:1. According to some embodiments, the molar ratio between a member of the aegerolysin family and a member of the MACPF superfamily is about 60:1. According to some embodiments, the molar ratio between a member of the aegerolysin family and a member of the MACPF superfamily is about 70:1. According to some embodiments, the molar ratio between a member of the aegerolysin family and a member of the MACPF superfamily is about 80:1. According to some embodiments, the molar ratio between a member of the aegerolysin family and a member of the MACPF superfamily is about 90:1. According to some embodiments, the molar ratio between a member of the aegerolysin family and a member of the MACPF superfamily is about 100:1. According to some embodiments, the molar ratio between a member of the aegerolysin family and a member of the MACPF superfamily is about 200:1. According to some embodiments, the molar ratio between a member of the aegerolysin family and a member of the MACPF superfamily is about 300:1. According to some embodiments, the molar ratio between a member of the aegerolysin family and a member of the MACPF superfamily is about 400:1. According to some embodiments, the molar ratio between a member of the aegerolysin family and a member of the MACPF superfamily is about 500:1. According to some embodiments, the molar ratio between a member of the aegerolysin family and a member of the MACPF superfamily is about 600:1. According to some embodiments, the molar ratio between a member of the aegerolysin family and a member of the MACPF superfamily is about 700:1. According to some embodiments, the molar ratio between a member of the aegerolysin family and a member of the MACPF superfamily is about 800:1. According to some embodiments, the molar ratio between a member of the aegerolysin family and a member of the MACPF superfamily is about 900:1. According to some embodiments, the molar ratio between a member of the aegerolysin family and a member of the MACPF superfamily is about 1000:1.

The composition may be applied at least once a week. For example, it may be applied 1 to 3 times a week, such as two times a week. The bi-component protein complex may be applied at least once a day. For example, it may be applied 1 to 3 times a day, such as twice a day.

The present invention also provides the use of a bi-component protein complex or a composition as described herein for the preparation of plant protection agent.

It is understood that all details provided herein with respect to the the bi-component protein complex, particularly with respect to the member of the aegerolysin family and the member of the MACPF superfamily, and plant pest, including all embodiments, apply mutatis mutandis to the use for preparation of a plant protection agent.

The present invention also provides a transgenic plant or progeny thereof which expresses or is capable of expressing a bi-component protein complex as described herein.

It is understood that all details provided herein with respect to the the bi-component protein complexes, particularly with respect to the member of the aegerolysin family and the member of the MACPF superfamily, including all embodiments, apply mutatis mutandis to the transgenic plant.

A transgenic plant or progeny thereof of the present invention suitably comprises (such as stably transformed with) one or more recombinant nucleic acid molecules (such as DNA) comprising nucleotide sequences that encode a bi-component protein complex as described herein, said nucleotide sequences being operably linked to at least one promoter that is functional in said plant cell to cause the production of mRNA molecules. The transgenic plant or progeny thereof may, for example, comprise one or more recombinant nucleic acid molecules (such as DNA) comprising a nucleotide sequence encoding the member of the aegerolysin family, and one or more recombinant nucleic acid molecules comprising a nucleotide sequence encoding the member of the MACPF superfamily, said nucleotide sequences being operably linked to at least one promoter that is functional in said plant cell to cause the production of mRNA molecules. The coding sequences may be comprised by the same or different recombinant nucleic acid molecules. Hence, the resulting mRNAs may be mono- or polycistronic.

The one or more recombinant nucleic acid molecules may be episomal (not contained within a chromosom) or may be stably integrated into a chromosome of the plant genome. According to certain embodiments, the one or more recombinant nucleic acid molecules may be episomal, such as in the form of a vector (such as in the form of an expression vector). According to certain embodiments, the one or more recombinant nucleic acid molecules are stably integrated into a chromosome of the plant genome.

A plant is transformed with a DNA molecule having genes encoding aegerolysin: ostreolysin A6 (OlyA6) or pleurotolysin A2 (PlyA2) or erylysin A (EryA) and pleurotolysin B (PlyB) and comprising an upstream and downstream regulatory sequence of these genes enabling the expression of said proteins in the transformed plant. The upstream regulatory sequence comprises promotor which can be constitutive (ex. CaMV 35S, Ubiquitin, Actin, EF1α, Nos, Tubulin, GAPDH, CAM, MAS, EF2 promotor) tissue-specific (ex. GSSP1, GSSP3, GSSP5, GSSP6, GSSP7, MT3-A, LC01, SynR2 SynR1, pCL), inducible, or synthetic. The proteins are expressed separately or as a fusion, linked with a peptide. Additionally, selectable marker genes, such as antibiotic resistance genes, are included to aid in the identification of successfully transformed cells. The DNA molecule is usually delivered into the plant cells via a vector, such as a plasmid or viral genome, to facilitate its integration into the plant genome.

The transformation of plant cells with an aegerolysin and PlyB genes may involve Agrobacterium-mediated transformation, biolistic (gene gun) transformation, or protoplast transformation.

The transformed plant may be grown next to control plants which have not been transformed and thus do not express OlyA6, PlyA2 or EryA and PlyB. At a certain development stage (halfway grown and active) the transformed plants and the control plants are screened to confirm the presence and expression of the genes of interest in the transformed plants, using molecular assays, such as PCR, Southern blotting, Western blotting. The next step involves exposure of the transformed plants directly to the pests such as Colorado potato beetle (Leptinotarsa decemlineata) or Western Corn rootworm (Diabrotica virgifera virgifera) in a controlled environment and their response is observed over time. Additionally, field trials are conducted to assess the performance of the transformed plants under real-world conditions. This involves planting the transformed plants alongside non-transformed controls in pest-infested fields and monitoring their growth and pest resistance over the course of a growing season.

Promoters useful in accordance with the invention are any known promoters that are functional in a plant cell to cause the production of an mRNA molecule. Many such promoters are known to the skilled person. The use of promoters for protein expression is generally known to those of skilled in the art of molecular biology, for example, see Sambrook et al., Molecular cloning: A Laboratory Manual, Cold Spring Harbor Laboratory, Cold Spring Harbor, N.Y., 1989. The promoter employed may be inducible or constitutive.

Non-limiting examples of plant functional promoters are the Lactuca sativa psbA promoter, the tobacco psbA promoter, the tobacco rrn16 PEP+NEP promoter, the CaMV 35S promoter, the 19S promoter, the tomato E8 promoter, the nos promoter, the Mac promoter, the pet E promoter or the ACT1 promoter.

The recombinant nucleic acid molecule(s) may further comprise at least one regulatory element selected from the group consisting of a 5′ untranslated region (5′ UTR), 3′ untranslated region (3′ UTR), and transit peptide region.

According to certain embodiments, the recombinant nucleic acid molecule is stably integrated into the genome of the transgenic plant or progeny thereof.

The transgenic plant may be (or derived from) any plant of interest. The transgenic plant may be an angiosperm or a gymnosperm. According to certain embodiments, the transgenic plant is an angiosperm. According to certain embodiments, the transgenic plant is a gymnosperm.

The transgenic plant may be a dicot or monocot. According to certain embodiments, the plant is a dicot. According to certain embodiment, the plant is a monocot.

The transgenic plant may be a food plant (i.e. a plant some parts of which provides food for animal or human consumption), such as fruit plant.

The transgenic plant may be a crop plant, such as a food crop plant. According to certain embodiments, the transgenic plant is a food crop plant such as a potato plant or maize plant. According to some embodiments, the transgenic plant is a potato plant, such as Solanum tuberosum. According to some embodiments, the plant is a maize plant. According to some embodiments, the plant is cabbage. According to some embodiments, the plant is asparagus.

### Certain Definitions

As used herein, »controlling a plant pest« or »plant pest control« means reducing or eliminating the plant pest, such as the pest insect.

As used herein, a “pesticide” is a compound or composition used for reducing or eliminating insects harmful to cultivated plants.

As used herein, “vector” refers to a nucleic acid molecule capable of transporting another nucleic acid molecule to which it has been linked. One type of vector is a “plasmid”, which refers to a circular double stranded nucleic acid loop into which additional nucleic acid segments can be ligated. Certain vectors are capable of directing the expression of genes to which they are operatively linked. Such vectors are referred to herein as “expression vectors”. Certain other vectors are capable of facilitating the insertion of a recombinant DNA molecule into a genome of a plant. Such vectors are referred to herein as “transformation vectors”. In general, vectors of utility in recombinant nucleic acid techniques are often in the form of plasmids. In the present specification, “plasmid” and “vector” can be used interchangeably as the plasmid is the most commonly used form of a vector. Large numbers of suitable vectors are known to those of skill in the art and commercially available.

As used herein, “promoter” refers to a sequence of DNA, usually upstream (5′) of the coding region of a structural gene, which controls the expression of the coding region by providing recognition and binding sites for RNA polymerase and other factors which may be required for initiation of transcription. The selection of the promoter will depend upon the nucleic acid sequence of interest. A “promoter functional in a plant cell” refers to a “promoter” which is capable of supporting the initiation of transcription in plant cells, enabling the synthesis of an mRNA molecule.

As used herein, “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. A promoter sequence is “operably-linked” to a gene when it is in sufficient proximity to the transcription start site of a gene to regulate transcription of the gene.

“% sequence identity” of an amino acid sequence to a reference amino acid sequence, as used herein, defines the % identity calculated from the two amino acid sequences as follows: The sequences are aligned using Version 9 of the Genetic Computing Group's GAP (global alignment program), using the default BLOSUM62 matrix with a gap open penalty of −12 (for the first null of a gap) and a gap extension penalty of −4 (for each additional null in the gap). After alignment, % identity is calculated by expressing the number of matches as a percentage of the number of amino acids in the reference amino acid sequence.

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### EXAMPLES

### Example 1

### Biological Assays

I. Feeding Bioassay with Potato Leaves Treated with Recombinant Proteins from the Fungal Genus Pleurotus to Assess Toxicity to CPB

### 1. Principle:

This bioassay was used to assess the impact of recombinant proteins from fungal genus Pleurotus on the survival of CPB larvae. We used potato leaves treated with recombinant proteins. The bioassay was carried out for 5 days. The survival rate was recorded daily. Weight measurements were recorded on day 1 and day 5.

### 2. Recombinant Proteins:

Proteins from the fungal genus Pleurotus, OlyA6, PlyA2, EryA and PlyB, were prepared in recombinant form by the use of heterologous expression system for protein production in Escherichia coli. Proteins were purified with chromatographic procedures and stored in buffer solutions at −20° C. Appropriate working protein mixtures, containing an aegerolysin and its protein partner, were prepared. The concentrations of fungal aegerolysin and PlyB were 0.5 mg/ml and 0.04 mg/mL, respectively.

### 3. Insects:

CPB larvae, collected from potato fields a day prior to the experiments, were divided into two groups: young larvae (L1+L2) and old larvae (L3+L4). In L1 and L2, the pronotum is entirely black. In L3, the anterior margin of the pronotum appears orange-brown. In L4, about half the pronotum is light brown anteriorly.

### 4. Procedure:

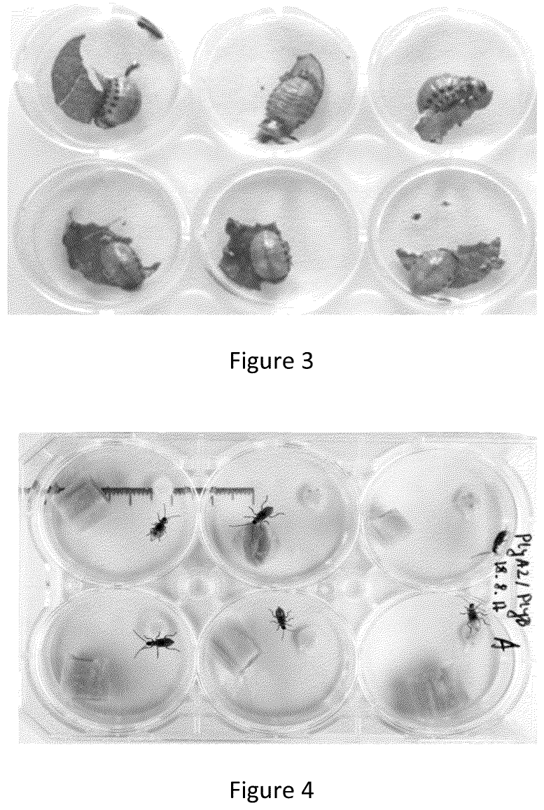

Potato leaf disks, 14 mm in diameter, were soaked in 5 mL of each protein mixture for 5 min, before being placed in 6-well microplate. A single CPB larva was transferred to each well (FIG. 3). Fresh potato disk leaves, treated with proteins, were added every second day. Three replicate 6-well plates per treatment were performed, and the experiment repeated twice independently, giving a total of 36 larvae for each treatment. The bioassay was performed in an incubator at 22° C.±1, RH 70% and a photoperiod of 16:8 (L:D). Buffer (20 mM Tris 0.5% glycerol pH 8.0) treated leaf disks were used as a negative control and insecticide Actara 25 WG (a.i. thiametoxam) treated leaf disks as a positive control. The time-based larval mortality was analyzed by Kaplan-Meier survival analysis and the effect of protein mixtures on larval weight change by Kruskal-Wallis test, followed by Dunn's post hoc test.

II. Feeding Bioassay with Artificial Diet for Adult WCR Mixed with Recombinant Proteins from the Fungal Genus Pleurotus to Assess Toxicity to WCR

### 1. Principle

This bioassay was used to assess the impact of recombinant proteins from fungal genus Pleurotus on the survival of WCR beetles. We used artificial diet mixed with recombinant proteins. The bioassay was carried out for 7 days. The survival rate was recorded daily.

### 2. Recombinant Proteins

Proteins from the fungal genus Pleurotus, OlyA6, PlyA2, EryA and PlyB, were prepared in recombinant form by the use of heterologous expression system for protein production Escherichia coli. Proteins were purified with chromatographic procedures and stored in buffer solutions at −20° C. Appropriate working protein mixtures, containing an aegerolysin and its protein partner, were prepared. The final concentrations of the fungal aegerolysin and PlyB in the mixture with artificial food were 0.5 mg/ml and 0.04 mg/mL, respectively.

### 3. Insects:

Beetles, used for the experiments, were collected from corn fields a day prior to the experiments.

### 4. Procedure:

An artificial diet (pH 7.0) comprising of: sucrose (33 g), alphacel (18.6 g), casein (15.9 g), soy flour (12.4 g), wheat germ (12.4 g), brewer's yeast (5 g), vitamin mix (Vanderzant, 1.2 g), salt mix (Wesson, 1.2 g), cholesterol (0.3 g), glycerol (11 mL) and dH₂O (82.5 mL), was mixed for WCR. 30 μL mixture, comprising of artificial diet and protein complexes (15 μl each) was pipetted to each well of a 6-well plate. A WCR adult was placed in each well (FIG. 4). Three replicate 6-well plates per treatment were performed, and the experiment repeated twice independently, giving a total of 36 adult insects for each treatment. The bioassay was performed in an incubator at 22° C.±1, RH 70% and a photoperiod of 16:8 (L:D). Buffer (20 mM Tris 0.5% glycerol pH 8.0) mixed with artificial food (1:1, v:v) was used as a negative control and 0.5% Decis (a.i. deltamethrin) mixture as a positive control.

### Biophysical Tests

### III. Surface Plasmon Resonance Measurements

### 1. Principle:

We used surface plasmon resonance for determining interactions of proteins from the fungal genus Pleurotus with artificial lipid vesicles containing CPE. We immobilized large unilamellar vesicles (LUV) containing CPE on the surface of the sensor chip. Solutions of the appropriate aegerolysin, alone or in combination with PlyB were injected across the surface of the sensor chip.

### 2. Reagents:

The interactions were monitored on Biacore X surface plasmon resonance (SPR)-based refractometer, using L1 chip. For immobilisation of LUVs composed of mixture of lipids (CPE: palmitoyl-2-oleoyl-sn-glycero-3-phosphocholine [POPC]:Chol [5:47.5:47.5, mol:mol:mol]) and as running buffer we used 20 mM Tris, 140 mM NaCl, 5 μM EDTA pH 7.4. As analytes we used recombinant proteins from the fungal genus Pleurotus: OlyA6, EryA, PlyA2 and PlyB. Experiments were performed at 25° C. The data were processed with BIAevaluation software (GE Healthcare).

### 3. Procedure:

After initial cleaning of the chip with regeneration solutions (sodium dodecyl sulphate [SDS] and octyl β-D-glucopyranoside [OG], 1-min injection, flow rate 10 μL/min), LUVs containing CPE were bound to the second flow cell of sensor chip L1 to reach response of approximately 7000 RU. The first flow cell was left empty and used to control the possible nonspecific binding of proteins to the dextran matrix. Looslely bound LUVs were washed from the surface with 1-min injection of 100 mM NaOH. The non-specific binding of the proteins was minimized with 1-minute injection of 0.1 mg/ml bovine serum albumin at flow rate 30 μL/min. Appropriate solutions of proteins and protein complexes were injected at a flow rate 10 μL/min. The interactions of OlyA6, PlyA2 or EryA with LUVs containing CPE were tested. Furthermore, we analysed the interactions of protein complexes OlyA6/PlyB, PlyA2/PlyB or EryA/PlyB with LUVs. Regeneration between injection was achieved with 1-min injection of 0.5% SDS and 40 mM OG with flow rate of 10 μL/min.

IV. Lytic Activity of Fungal Aegerolysins with their Partner PlyB

### 1. Principle:

We used calcein-loaded small unilamellar vesicles (SUVs) containing CPE in order to confirm the lytic activity of OlyA6, PlyA2 or EryA with their protein partner PlyB.

### 2. Reagents:

The lytic activity of the protein complexes was assayed in a fluorescence microplate reader at 25° C. We used calcein-loaded SUVs composed of mixture of lipids (CPE:POPC:Chol [5:47.5:47.5, mol:mol:mol]).

### 3. Procedure:

Protein mixtures comprising fungal aegerolysins OlyA6, PlyA2 or EryA and PlyB (12.5:1, mol:mol) were dispensed into a multi-well microplate at the following concentrations: 10 μg/mL OlyA6, PlyA2 or EryA combined with 0.8 μg/mL PlyB. Calcein-loaded SUVs were added to the protein mixtures (5 μg/mL). The SUVs were excited at 485 nm and the intensity of the emitted fluorescence of released calcein was monitored at 535 nm for 30 min with 20 s intervals. The intensities of the emitted fluorescence at t=0 and t=30 were determined as F₀ and F, respectively SUVs were fully lysed with 1 mM Triton X-100, and measured fluorescence was determined as maximal fluorescence intensity (Fₘₐₓ). The percentage of calcein release R (%) was calculated as:

$$R ( \% ) = \frac{F - F o}{F max - F o} * 100$$

### Results

I. CPB Feeding Bioassay with Potato Leaves Treated with Recombinant Proteins from Fungal Genus Pleurotus

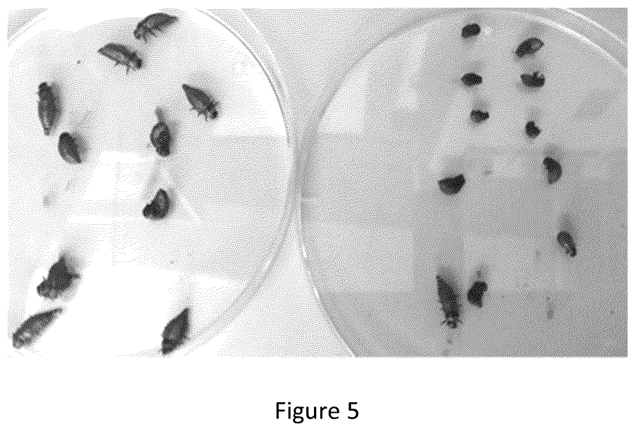

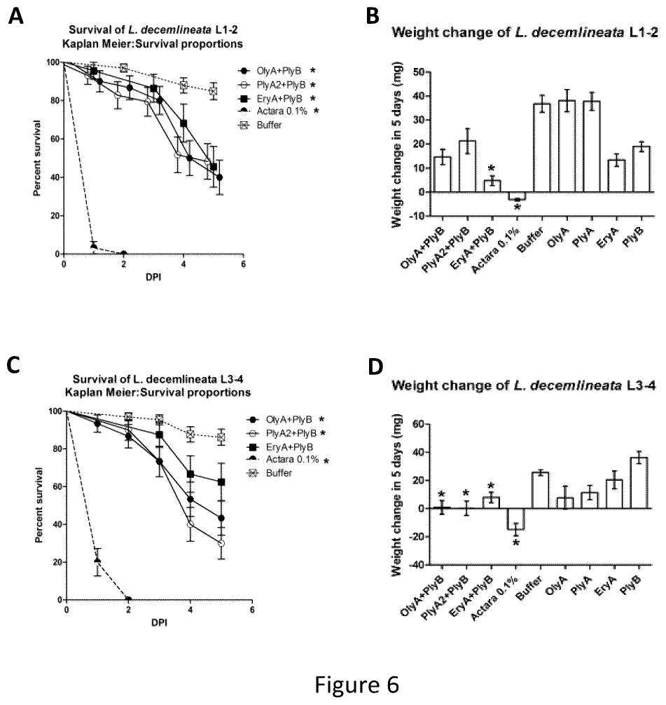

The effect of bi-component pesticidal complexes, comprising fungal aegerolysin and PlyB on the CPB larvae was tested. Larval mortality and body weight were analyzed. Exposure of CPB larvae to leaf disks treated with OlyA6/PlyB or PlyA2/PlyB protein mixtures caused significant larval mortality on day 5 after initiation of feeding in both groups (FIGS. 5-6). EryA/PlyB protein mixture caused significant larval mortality also in young larvae (L1+L2). The feeding behavior of exposed larvae was significantly different from that of control larvae (FIGS. 5-6). After day 1, exposed larvae showed decrease in food consumption. L1+L2 treated with EryA/PlyB showed a significantly lower weight increase compared with the weights of control larvae, whereas all protein mixtures significantly reduced larval weight increase in the case of L3+L4. Individual aegerolysins (OlyA6, PlyA2 or EryA) or only the component B (PlyB) alone did not show a significant effect on larval weight change in L1+L2 or L3+L4.

II. Feeding Bioassay with Artificial Diet Mixed with Recombinant Proteins from Fungal Genus Pleurotus Targeting Adult WCR

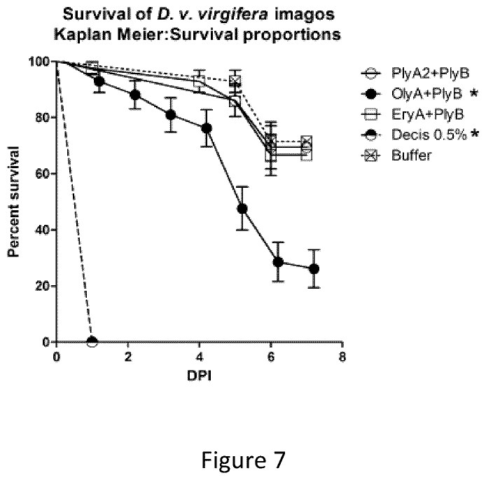

The effect of bi-component pesticidal complexes, comprising fungal aegerolysin and PlyB on the WCR beetles was tested. Insect mortality was evaluated on a daily basis. Exposure of WCR to artificial food mixed with OlyA6/PlyB resulted in significant increase in mortality on day 5 after the initiation of feeding. The feeding behaviour of exposed larvae was different from that of control larvae. After day 4, exsposed larvae showed decrease in food consumption (FIG. 7).

### III. Surface Plasmon Resonance Measurements

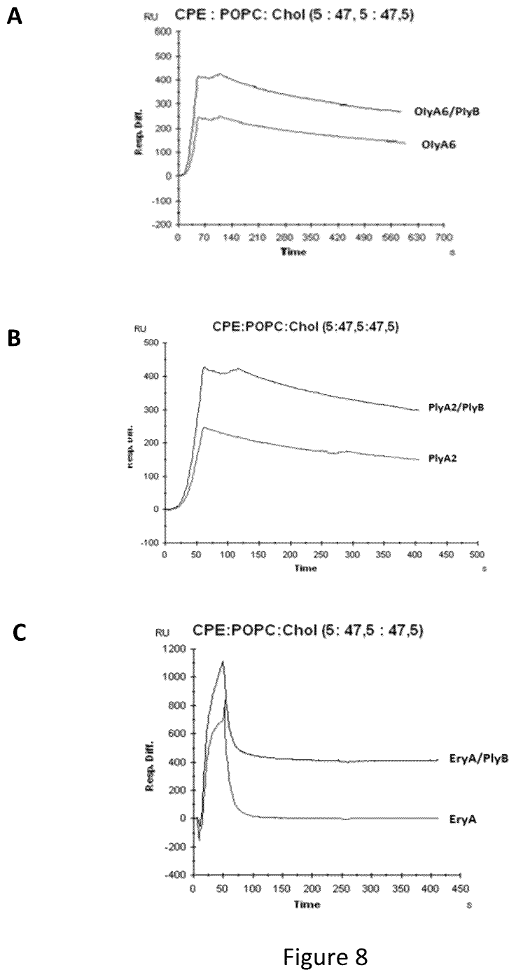

We monitored the binding of fungal aegerolysins OlyA6, PlyA2 or EryA (with or without pleurotolysin B) to large unilamellar vesicles containing CPE using surface plasmon resonance. The results show clearly that all tested aegerolysins specifically interact with CPE-containing artificial membranes, and that this interaction is stabilized in the presence of PlyB (FIG. 8).

IV. Lytic Activity of Fungal Aegerolysins with their Partner PlyB

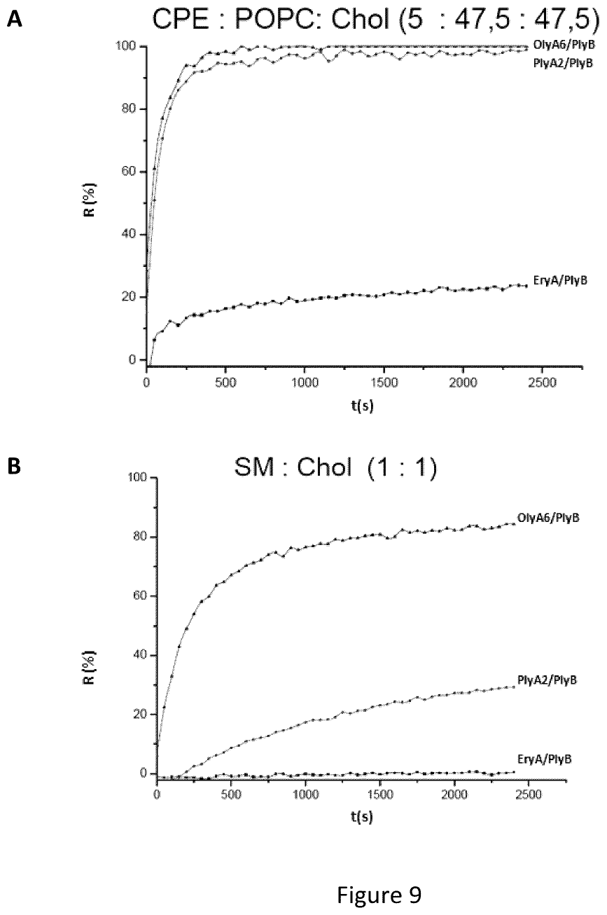

The results show that artificial membranes were permeabilized with aegerolysins OlyA6, PlyA2 or EryA in combination with PlyB, if the membranes contained CPE. Protein complexes OlyA6/PlyB and PlyA2/PlyB are more lytic than EryA/PlyB (FIG. 9).

### Example 2

### Preparing a Transformed Maize

A maize is transformed with a DNA molecule having genes encoding an aegerolysin and PlyB, following the protocol described in the references below:
Yalpani N, Altier D, Barry J, Kassa A, Nowatzki T M, Sethi A, Zhao J Z, Diehn S, Crane V, Sandahl G, Guan R, Poland B, Perez Ortega C, Nelson M E, Xie W, Liu L, Wu G. An Alcaligenes strain emulates Bacillus thuringiensis producing a binary protein that kills corn rootworm through a mechanism similar to Cry34Ab1/Cry35Ab1. Sci Rep. 2017 Jun. 8; 7(1):3063. doi: 10.1038/s41598-017-03544-9.
Mi X, Ji X, Yang J, Liang L, Si H, Wu J, Zhang N, Wang D. Transgenic potato plants expressing cry3A gene confer resistance to Colorado potato beetle. C R Biol. 2015 July; 338(7):443-50. doi: 10.1016/j.crvi.2015.04.005

The maize plant is transformed with a DNA molecule having genes encoding the PlyA2 and PlyB, and comprising a promoter upstream of these two genes enabling the expression of said two proteins in the transformed plant.

Plant transformation is performed by delivery of the DNA into a single cell from the plant, and then regeneration into full fertile plants expressing PlyA2 and PlyB. The delivery is performed using Agrobacterium.

### Example 3

Assaying a Plant for being Resistant to a Plant Pest

To evaluate the potential damage caused by Western corn rootworm colonies on plants, we employ a method known as the Root Trainer assay. This assay is detailed in the references provided below.
Yalpani N, Altier D, Barry J, Kassa A, Nowatzki T M, Sethi A, Zhao J Z, Diehn S, Crane V, Sandahl G, Guan R, Poland B, Perez Ortega C, Nelson M E, Xie W, Liu L, Wu G. An Alcaligenes strain emulates Bacillus thuringiensis producing a binary protein that kills corn rootworm through a mechanism similar to Cry34Ab1/Cry35Ab1. Sci Rep. 2017 Jun. 8; 7(1):3063. doi: 10.1038/s41598-017-03544-9.
Schellenberger U, Oral J, Rosen B A, Wei J Z, Zhu G, Xie W, McDonald M J, Cerf D C, Diehn S H, Crane V C, Sandahl G A, Zhao J Z, Nowatzki T M, Sethi A, Liu L, Pan Z, Wang Y, Lu A L, Wu G, Liu L. A selective insecticidal protein from Pseudomonas for controlling corn rootworms. Science. 2016 Nov. 4; 354(6312):634-637. doi: 10.1126/science.aaf6056.

To assess the resistance of transgenic potatoes to the Colorado potato beetle, we investigate the insects' leaf consumption, insect biomass accumulation, and mortality using protocols outlined in the references provided below.
Mi X, Ji X, Yang J, Liang L, Si H, Wu J, Zhang N, Wang D. Transgenic potato plants expressing cry3A gene confer resistance to Colorado potato beetle. C R Biol. 2015 July; 338(7):443-50. doi: 10.1016/j.crvi.2015.04.005
Zhou Z, Pang J, Guo W, Zhong N, Tian Y, Xia G, Wu J. Evaluation of the resistance of transgenic potato plants expressing various levels of Cry3A against the Colorado potato beetle (Leptinotarsa decemlineata Say) in the laboratory and field. Pest Manag Sci. 2012 December; 68(12):1595-604. doi: 10.1002/ps.3356.

### REFERENCES

Alyokhin, A., Baker, M., Mota-Sanchez, D., Dively, G., Grafius, E. (2008). Colorado Potato Beetle Resistance to Insecticides. American Journal Of Potato Research, 85(6), 395-413. http://dx.doi.org/10.1007/s12230-008-9052-0
Alyokhin, A., Mota-Sanchez, D., Baker, M., Snyder, W., Menasha, S., Whalon, M. et al. (2014). The Red Queen in a potato field: integrated pest management versus chemical dependency in Colorado potato beetle control. Pest Management Science, 71(3), 343-356. http://dx.doi.org/10.1002/ps.3826
Anderluh, G., Kisovec, M., Kraševec, N., Gilbert, R. J. (2014). Distribution of MACPF/CDC proteins. Subcellular Biochemistry, 80, 7-30
Berne, S., Lah, L., Sepčić, K. (2009). Aegerolysins: Structure, function, and putative biological role. Protein Science, 18(4), 694-706. http://dx.doi.org/10.1002/pro.85
Bhat, H., Ishitsuka, R., Inaba, T., Murate, M., Abe, M., Makino, A. et al. (2015). Evaluation of aegerolysins as novel tools to detect and visualize ceramide phosphoethanolamine, a major sphingolipid in invertebrates. The FASEB Journal, 29(9), 3920-3934. http://dx.doi.org/10.1096/fj.15-272112
Butala, M., Novak, M., Kraševec, N., Skočaj, M., Veranič, P., Maček, P., Sepčić, K. (2017). Aegerolysins: Lipid-binding proteins with versatile functions. Seminars In Cell And Developmental Biology. http://dx.doi.org/10.1016/j.semcdb.2017.05.002
Casagrande, RA. (1987). The colorado Potato Beetle: 125 Years of mismanagement. American Entomologist, 33(3), 142-150. https://doi.org/10.1093/besa/33.3.142
Chu, C., Sun, W., Spencer, J., Pittendrigh, B., Seufferheld, M. (2014). Differential effects of RNAi treatments on field populations of the western corn rootworm. Pesticide Biochemistry And Physiology, 110, 1-6. http://dx.doi.org/10.1016/j.pestbp.2014.02.003
Crone, H. D., Bridges, R G. (1963). The phospholipids of the housefly, Musca domestica. Biochemical Journal, 89, 11-21
Devine, G., Furlong, M. (2007). Insecticide use: Contexts and ecological consequences. Agriculture And Human Values, 24(3), 281-306. http://dx.doi.org/10.1007/s10460-007-9067-z
Gassmann, A. (2012). Field-evolved resistance to Bt maize by western corn rootworm: Predictions from the laboratory and effects in the field. Journal Of Invertebrate Pathology, 110(3), 287-293. http://dx.doi.org/10.1016/j.jip.2012.04.006
Gassmann, A., Petzold-Maxwell, J., Keweshan, R., Dunbar, M. (2011). Field-Evolved Resistance to Bt Maize by Western Corn Rootworm. Plos ONE, 6(7), e22629. http://dx.doi.org/10.1371/journal.pone.0022629
Itasaka, O., Hori, T., Uno, A., Iwamori, M. (1973). Occurrence of of Ceramide Phosphorylethanolamine Containing Hydroxy Fatty Acid in a Bivalve. Journal of Biochemistry, 73, 191-193.
Jakka, S., Shrestha, R., Gassmann, A. (2016). Broad-spectrum resistance to Bacillus thuringiensis toxins by western corn rootworm (Diabrotica virgifera virgifera). Scientific Reports, 6(1). http://dx.doi.org/10.1038/srep27860
Kaiser-Alexnat, R. (2009). Protease activities in the midgut of Western corn rootworm (Diabrotica virgifera virgifera LeConte). Journal Of Invertebrate Pathology, 100(3), 169-174. http://dx.doi.org/10.1016/j.jip.2009.01.003
Kelker, M., Berry, C., Evans, S., Pai, R., McCaskill, D., Wang, N. et al. (2014). Structural and Biophysical Characterization of Bacillus thuringiensis Insecticidal Proteins Cry34Ab1 and Cry35Ab1. Plos ONE, 9(11), e112555. http://dx.doi.org/10.1371/journal.pone.0112555
Kuhlmann, U., Burgt, W. A. C. M., 1998. Possibilities for biologicalcontrol of the western corn rootworm, Diabrotica virgiferavirgifera LeConte, in Central Europe. Biocontrol News And Information, 19, 59-68
Ludwick, D., Meihls, L., Ostlie, K., Potter, B., French, L., Hibbard, B. (2017). Minnesota field population of western corn rootworm (Coleoptera: Chrysomelidae) shows incomplete resistance to Cry34Ab1/Cry35Ab1 and Cry3Bb1. Journal Of Applied Entomology, 141(1-2), 28-40. http://dx.doi.org/10.1111/jen.12377
Lukoyanova, N., Kondos, S. C., Farabella, I., Law, R. H. P., Reboul, C. F., Caradoc-Davies, T. T., Spicer, B., Kleifeld, O., Traore, D. A. K., Ekkel, S. M., Voskoboinik, I., Trapani, J. A., Hatfaludi, T. Z., Oliver, K., Hotze, E. M., Tweten, R. K., Whisstock, J. C., Topf, M., Saibil, H. R., Dunstone, M. A. (2015). Conformational changes during pore formation by the perform-related protein pleurotolysin, Plos Biology, 13 (2), 1-15. http://dx.doi.org/10.1371/journal.pbio.1002049
Masson, L., Schwab, G., Mazza, A., Brousseau, R., Potvin, L., Schwartz, J. (2004). A Novel Bacillus thuringiensis (PS149B1) Containing a Cry34Ab1/Cry35Ab1 Binary Toxin Specific for the Western Corn Rootworm Diabrotica virgifera virgiferaLeConte Forms Ion Channels in Lipid Membranes. Biochemistry, 43(38), 12349-12357. http://dx.doi.org/10.1021/bi048946z
Meinke, L. J., Siegfried, B. D., Wright, R. J., Chandler, L. D. (1998). Adult Susceptibility of Nebraska Western Corn Rootworm (Coleoptera: Chrysomelidae) Populations to Selected Insecticides. Journal of Economic Entomology, 91(3), 594-600
Meissle, M., Pilz, C., Romeis, J. (2009). Susceptibility of Diabrotica virgifera virgifera (Coleoptera: Chrysomelidae) to the Entomopathogenic Fungus Metarhizium anisopliae when Feeding on Bacillus thuringiensis Cry3Bb1-Expressing Maize. Applied And Environmental Microbiology, 75(12), 3937-3943. http://dx.doi.org/10.1128/aem.00432-09
Novak, M., Kraševec, N., Skočaj, M., Maček, P., Anderluh, G., Sepčić, K. (2014). Fungal aegerolysin-like proteins: distribution, activities, and applications. Applied Microbiology And Biotechnology, 99(2), 601-610. http://dx.doi.org/10.1007/s00253-014-6239-9
Ota, K., Leonardi, A., Mikelj, M., Skočaj, M., Wohlschlager, T., Künzler, M. et al. (2013). Membrane cholesterol and sphingomyelin, and ostreolysin A are obligatory for pore-formation by a MACPF/CDC-like pore-forming protein, pleurotolysin B. Biochimie, 95(10), 1855-1864. http://dx.doi.org/10.1016/j.biochi.2013.06.012
Ota, K., Butala, M., Viero, G., Dalla Serra, M., Sepčič, K., Maček, P. (2014). Fungal MACPF-like proteins and aegerolysins: bi-component pore-forming proteins? Subcellular Biochemistry, 80, 271-91.
Pereira, A., Wang, H., Zukoff, S., Meinke, L., French, B., Siegfried, B. (2015). Evidence of Field-Evolved Resistance to Bifenthrin in Western Corn Rootworm (Diabrotica virgifera virgifera LeConte) Populations in Western Nebraska and Kansas. PlosS ONE, 10(11), e0142299. http://dx.doi.org/10.1371/journal.pone.0142299
Perlak, F. J., Stone, B. T., Muskopf, Y. M., Petersen, L. J., Parker, G. B., McPherson, S. A., Wyman, J., Love, S., Reed, G., Biever, D., Fischohoff. (1993). Genetically improved potatos: protection from damage by Colorado potato beetles. Plant Molecular Biology, 22(2), 313-321
Qureshi, N., Chawla, S., Likitvivatanavong, S., Lee, H., Gill, S. (2014). The Cry Toxin Operon of Clostridium bifermentans subsp. malaysia Is Highly Toxic to Aedes Larval Mosquitoes. Applied And Environmental Microbiology, 80(18), 5689-5697. http://dx.doi.org/10.1128/aem.01139-14
Sambrook, J., Fritsch, F. E., and Maniatis, T. (1989) Molecular cloning: A Laboratory Manual. Second edition. New York: Cold Spring Harbor Laboratory Press.
Sepčić, K., Berne, S., Rebolj, K., Batista, U., Plemenitaš, A., Šentjurc, M., Maček, P. (2004). Ostreolysin, a pore-forming protein from the oyster mushroom, interacts specifically with membrane cholesterol-rich lipid domains. FEBS Letters, 575(1-3), 81-85. http://dx.doi.org/10.1016/j.febslet.2004.07.093
Shibata, T., Kudou, M., Hoshi, Y., Kudo, A., Nanashima, N., Miyairi, K. (2010). Isolation and characterization of a novel two-component hemolysin, erylysin A and B, from an edible mushroom, Pleurotus eryngii. Toxicon, 56(8), 1436-1442. http://dx.doi.org/10.1016/j.toxicon.2010.08.010
Skočaj, M., Resnik, N., Grundner, M., Ota, K., Rojko, N., Hodnik., V. et al. (2014). Tracking Cholesterol/Sphingomyelin-Rich Membrane Domains with the Ostreolysin A-mCherry Protein. Plos ONE, 9(3), e92783. http://dx.doi.org/10.1371/journal.pone.0092783
Tomita, T., Noguchi, K., Mimuro, H., Ukaji, F., Ito, K., Sugawara-Tomita, N., Hashimoto, Y. (2004). Pleurotolysin, a Novel Sphingomyelin-specific Two-component Cytolysin from the Edible Mushroom Pleurotus ostreatus, Assembles into a Transmembrane Pore Complex. Journal Of Biological Chemistry, 279(26), 26975-26982. http://dx.doi.org/10.1074/jbc.m402676200
Tu, J., Zhang, G., Datta, K., Xu C., He, Y., Zhang, Q., Khush, S., Datta, S. K. (2000). Field performance of transgenic elite commercial hybrid rice expressing Bacillus thuringiensis δ-endotoxin. Nature, 18, 1101-1104. http://dx.doi.org/10.1038/80310
Vacaru, A. M., van den Dikkenberg, J., Terne, s P., Holthuis, J. C. (2013). Ceramide phosphoethanolamine biosynthesis in Drosophila is mediated by a unique ethanolamine phosphotransferase in the Golgi lumen. The Journal of Biological Chemistry, 288(16), 11520-30. http://dx.doi.org/10.1074/jbc.M113.460972
Yalpani, N., Altier, D., Barry, J., Kassa, A., Nowatzki, T., Sethi, A. et al. (2017). An Alcaligenes strain emulates Bacillus thuringiensis producing a binary protein that kills corn rootworm through a mechanism similar to Cry34Ab1/Cry35Ab1. Scientific Reports, 7(1). http://dx.doi.org/10.1038/s41598-017-03544-9

1. A method for protecting a plant against a plant pest and/or controlling or treating a plant pest, comprising the step of: transforming said plant by introducing one or more nucleic acid construct(s) encoding a bi-component protein complex in a plant genome, consisting of a plurality of molecules selected from the group consisting of ostreolysin and pleurotolysin A and a plurality of molecules of pleurotolysin B (PlyB).

2. The method according to claim 1, wherein the transformed plant is subjected to an assay for verifying that said transformed plant has resistance against said plant pest.

3. The method according to claim 2, wherein said assay comprises growing the transformed plant and the control plant not being transformed, and then contacting both plants with said plant pest to assess the difference in resistance to said plant pest.

4. The method according to claim 2, wherein said assay comprises growing the transformed plants and then contacting half of the plants with said plant pest to assess the difference in resistance to said plant pest.

5. The method according to claim 1, wherein the ostreolysin is a polypeptide comprising an amino acid sequence having at least 95% sequence identity with SEQ ID NO: 1.

6. The method according to claim 1, wherein the pleurotolysin A is a polypeptide comprising an amino acid sequence having at least 95% sequence identity with SEQ ID NO: 2.

7. The method according to claim 1, wherein pleurotolysin B is a polypeptide comprising an amino acid sequence having at least 95% sequence identity with SEQ ID NO: 4.

8. The method according to claim 1, wherein the plant pest is an insect of the family Chrysomelidae.

9. The method according to claim 1, wherein the plant pest is an insect.

10. The method according to claim 1, wherein the plant pest is a larva of the insect or an imago of the insect.

11. The method according to claim 9, wherein the insect is of the genus Leptinotarsa.

12. The method according to claim 9, wherein the insect is Leptinotarsa decemlineata (Colorado potato beetle).

13. The method according to claim 9, wherein the insect is of the genus Diabrotica.

14. The method according to claim 11, wherein the insect is Diabrotica virgifera virgifera (Western corn rootworm).

15. The method according to claim 9, wherein the insect is selected from the group consisting Leptinotarsa decemlineata (Colorado potato beetle) and Diabrotica virgifera virgifera (Western corn rootworm).

16. The method according to claim 1, wherein the plant is a crop plant.

17. The method according to claim 1, wherein the plant is a potato plant.

18. The method according to claim 1, wherein the plant is a maize plant.

19. A transgenic plant or progeny thereof which expresses or is capable of expressing a bi-component protein complex as defined in claim 1.

20. The transgenic plant or progeny thereof according to claim 19, wherein said plant was transformed by introducing one or more nucleic acid construct(s) encoding a bi-component protein complex in a plant genome, consisting of a plurality of molecules selected from the group consisting of ostreolysin and pleurotolysin A and a plurality of molecules of pleurotolysin B (PlyB), so as to obtain said transgenic plant or progeny thereof.
